(19) United States
(12) Patent Application Publication
(10) Pub. No.: US 2025/0046409 A1
(43) Pub. Date: Feb. 6, 2025

(54) SYSTEMS AND METHODS FOR PHENOTYPING USING LARGE LANGUAGE MODEL PROMPTING

(71) Applicant: Tempus AI, Inc., Chicago, IL (US)

(72) Inventors: William E. Thompson IV, Denver, CO (US); Riccardo Miotto, New York, NY (US); David Michael Vidmar, Amherst, MA (US); RuiJun Chen, Livingston, NJ (US); Gabriel Alexander Parlato-Altay, Washington, DC (US)

(21) Appl. No.: 18/782,994

(22) Filed: Jul. 24, 2024

Related U.S. Application Data
(60) Provisional application No. 63/587,409, filed on Oct. 2, 2023.
(60) Provisional application No. 63/515,530, filed on Jul. 25, 2023.

(51) Int. Cl. G16H 10/60

(52) U.S. Cl. CPC G16H 10/60

(57) ABSTRACT

The application includes systems and methods for phenotyping. A request is received to identify a target population having a phenotype. Predefined characteristics associated with the phenotype are obtained. Using a retriever model, a set of subjects is identified as potential members of the target population by searching databases. Medical information is obtained by searching a second set of one or more databases and providing corresponding information to an artificial intelligence (AI) component. Natural language instructions are provided to the AI component to provide context to the AI component to determine whether a respective subject in the set of subjects has at least one of the one or more predefined characteristics. A subset of subjects is identified by the AI system to be, or to have a high likelihood of being, a member of the target population.

### RELATED APPLICATIONS

This application claims priority to U.S. Prov. App. No. 63/515,530, filed on Jul. 25, 2023, and entitled “Systems and Methods for Phenotyping using Large Language Model Prompting,” and to U.S. Prov. App. No. 63/587,409, filed on Oct. 2, 2023, and entitled “Systems and Methods for Phenotyping using Large Language Model Prompting,” each of which is hereby incorporated by reference in its entirety.

### TECHNICAL FIELD

The disclosed embodiments relate generally to systems and methods of phenotyping, patient discovery, and feasibility, including but not limited to phenotyping utilizing artificial intelligence and large language model prompting for phenotyping, patient discovery, and/or feasibility.

### BACKGROUND

Electronic health record (EHR) phenotyping in observational clinical research varies in methodology and quality, and often suffers from a number of limitations. Many studies utilize diagnosis codes such as ICD-9, ICD-10, or SNOMED codes without validating the quality of these codes to determine how well they represent their desired cohort of interest. Within studies utilizing claims data, there is limited ability to perform detailed chart reviews or manual abstraction for the development of phenotypes or code-based cohort definitions, although some population and cohort-level metrics may be used in aggregate to ascertain if the cohort seems grossly appropriate from a clinical perspective.

Many studies do not perform cohort characterization or chart review validation, instead trusting that codes primarily developed for non-research purposes (most often billing purposes) represent exactly what the code description says they should represent. However, modern coding in EHR systems is messy, incomplete, and often incorrect. This may be due to (i) design flaws, (ii) changes in a patient's clinical status and working diagnosis (e.g., as they proceed along a diagnostic pathway or by accident), and/or (iii) busy clinical schedules or data entry or mapping errors. Codes for either common diseases or those linked with high levels of reimbursement may be accurately captured, but other codes may be inaccurate or erroneous. The same issues exist for procedure codes, problem lists (some institutions update them regularly, some ignore them altogether), medications, and most other sources of EHR data. Based on practice patterns or geographic regions, codes to best capture the same disease may change dramatically, making code-based phenotypes limited in their generalizability.

### SUMMARY

Disease phenotyping within electronic health records (EHRs) involves identifying ground truth diagnoses in a patient's clinical history. These phenotypes play a crucial role in several essential functions, such as selecting patient groups for observational studies or interventional quality initiatives to close gaps in care, defining inclusion and exclusion criteria, and providing labels for subsequent modeling tasks (e.g., ECG-based prediction models). Relevant information for disease diagnosis may be scattered across different data sources in EHRs, including physician's free-text notes, the presence of International Classification of Diseases 9th and 10th revision (ICD-9/10) codes, prescribed medications, or laboratory values from medical procedures and tests. Moreover, this information is often inaccurate, which makes identification of true disease diagnosis even more challenging.

The ideal process involves subject matter experts (SMEs) manually reviewing patient files to determine disease diagnosis. Yet chart reviews are time-consuming, taking an average of 30 minutes per file. To address this, SMEs often create custom rules-based algorithms, combining ICD codes, laboratory values, medications, and procedures, to identify diseases. However, challenges arise, including coding errors, reporting biases, and data sparsity, requiring iterative refinement through a human-in-the-loop process. Scalability is hindered, especially when features from one EHR system do not generalize to others. Mapping rare diseases to common ontologies like SNOMED can also be problematic due to expert disagreements.

Machine learning approaches to phenotyping, both supervised and unsupervised, have shown varying degrees of promise. Supervised learning approaches often require high-quality labels and are therefore constrained by a labeling bottleneck. While unsupervised learning approaches circumvent this problem, they often are difficult to tailor to fit a particular disease definition or achieve certain acceptance criteria. The majority of work on phenotyping also mostly focuses on structured data and ignores clinical text. Yet, clinical notes often contain a superset of information found in the patient's structured EHR, and incorporating the notes holds the possibility of developing a better phenotype.

Large language models (LLMs) provide a great opportunity to interact efficiently and effectively with free text, without the need for labeled data or development of ad-hoc models. While prior work has explored using LLMs for phenotyping diseases, due to computational constraints imposed by LLMs such work has utilized only specific portions of the full patient record (e.g., discharge summaries or extracted counseling sections). This can be suboptimal for certain diseases and real-world data (RWD) which may have relevant information scattered across various sections and types of clinical documents.

In some embodiments, the disclosure addresses these limitations within practical computational constraints through use of a retrieval-augmented generative (RAG) approach to zero-shot phenotyping using LLMs. In some embodiments, the methods and systems described herein apply a RAG approach to process entire patient records with LLMs, e.g., as opposed to focusing solely on a specific type of clinical note. In some embodiments, this approach is used to analyze all clinical mentions throughout a patient's entire record without the need for predefined sections of interest.

In some embodiments, the disclosure provides a map-reduce paradigm for parallel snippet evaluation and resolution of potentially conflicting information during the output aggregation stage. This is advantageous given the substantial volume of potentially relevant information retrieved and use of the language model's reasoning abilities rather than relying on an intricate retrieval mechanism. In the disclosure below, the performance of this approach is assessed using the expertise of a physician subject matter expert to assist in developing a competing rules-based model, which is still the commonly used approach in healthcare practice and industry. Both models are then evaluated using an unseen test dataset, with our chosen disease phenotype being pulmonary hypertension (PH). Advantageously, the disclosed method significantly outperforms physician logic rules (F1 score of 0.62 vs. 0.75).

Accordingly, in some embodiments, the present disclosure describes systems and methods for using generative artificial intelligence (AI), such as large language models, to perform phenotyping. For example, phenotyping via large language model (LLM) prompting as described herein may involve one or more subject matter experts (SMEs) iterating directly on a set of natural language instructions to instruct an LLM to identify a subject having a disease (e.g., analogous to teaching a resident). Phenotyping via LLM prompting can circumvent the SME knowledge translation problem, does not require training a machine learning (ML) model (e.g., is zero-shot), and may dramatically improve phenotype development time (e.g., time-to-market).

In accordance with some embodiments, of a method of phenotyping includes (i) receiving a request to identify a target population having one or more predefined characteristics; (ii) identifying (e.g., using a retriever component) a set of subjects as potential members of the target population; (iii) obtaining (e.g., using the retriever component) medical information for the set of subjects by searching one or more databases; (iv) providing the medical information to an artificial intelligence (AI) component (e.g., that includes a large language model); (v) providing a set of natural language instructions to the AI component, where the set of natural language instructions instruct the AI component how to determine if a subject belongs to the target population; (vi) obtaining, from the AI component, identification of a subset of subjects from the set of subjects, the subset of subjects determined by the generative AI component to be members of the target population; and (vii) providing the identification of the subset of subjects to a user.

In accordance with some embodiments, a computing system is provided, such as a cloud computing system, a server system, a personal computer system, or other electronic device. The computing system includes control circuitry and memory storing one or more sets of instructions. The one or more sets of instructions including instructions for performing any of the methods described herein.

In accordance with some embodiments, a non-transitory computer-readable storage medium is provided. The non-transitory computer-readable storage medium stores one or more sets of instructions for execution by a computing system. The one or more sets of instructions including instructions for performing any of the methods described herein.

Thus, devices and systems are disclosed with methods for phenotyping. Such methods, devices, and systems may complement or replace conventional methods, devices, and systems for phenotyping.

The features and advantages described in the specification are not necessarily all-inclusive and, in particular, some additional features and advantages will be apparent to one of ordinary skill in the art in view of the drawings, specification, and claims provided in this disclosure. Moreover, it should be noted that the language used in the specification has been principally selected for readability and instructional purposes and has not necessarily been selected to delineate or circumscribe the subject matter described herein.

### BRIEF DESCRIPTION OF THE DRAWINGS

So that the present disclosure can be understood in greater detail, a more particular description can be had by reference to the features of various embodiments, some of which are illustrated in the appended drawings. The appended drawings, however, merely illustrate pertinent features of the present disclosure and are therefore not necessarily to be considered limiting, for the description can admit to other effective features as the person of skill in this art will appreciate upon reading this disclosure.

FIG. 1 is a block diagram illustrating an example platform in accordance with some embodiments.

FIG. 2 is a block diagram illustrating an example client device in accordance with some embodiments.

FIG. 3 is a block diagram illustrating an example server system in accordance with some embodiments.

FIG. 4 is a block diagram illustrating example databases in accordance with some embodiments.

FIGS. 5A, 5B, 5C, and 5D collectively are block diagrams illustrating example patient discovery processes in accordance with some embodiments.

FIG. 6 is a flow diagram illustrating an example method of phenotyping in accordance with some embodiments.

FIG. 7 details logical operations that may be used to combine filters in accordance with some embodiments.

FIG. 8 is a block diagram illustrating an example LLM-based phenotype architecture in accordance with some embodiments.

FIG. 9 shows an example structured phenotype developed for pulmonary hypertension in accordance with some embodiments.

FIGS. 10A-10B provide example descriptions and statistics of the various prompt templates in accordance with some embodiments.

FIG. 11 illustrates an example of a zero-shot prompt asking an LLM to assess whether a patient has PH based on the provided context in accordance with some embodiments.

In accordance with common practice, the various features illustrated in the drawings are not necessarily drawn to scale, and like reference numerals can be used to denote like features throughout the specification and figures.

### DETAILED DESCRIPTION

Identifying disease phenotypes from electronic health records (EHRs) is critical for numerous secondary uses such as clinical research and population health management. Manually encoding physician knowledge into rules, a common approach, becomes particularly challenging for rare diseases due to inadequate EHR coding, necessitating detailed review of clinical notes. Large language models (LLMs) offer promise in text understanding but might not efficiently handle the vast clinical documentation of real-world healthcare facilities. In one embodiment, the disclosure addresses this need by providing a zero-shot LLM-based method enriched by retrieval-augmented generation (RAG) and map-reduce, which pre-identifies disease-related text snippets to be used in parallel as queries for the LLM to establish diagnosis.

Advantageously, this method significantly outperforms use of physician logic rules. For example, as described herein, as applied to the problem of identifying pulmonary hypertension (PH), a rare disease characterized by elevated arterial pressures in the lungs, the disclosed method significantly outperforms physician logic rules (F1 score of 0.62 vs. 0.75). This method has the potential, for example, to enhance rare disease cohort identification, expanding the scope of robust clinical research and care gap identification.

The results presented in the Examples below underscore the potential of employing an LLM-based architecture to identify diseases across clinical notes. Unlike existing literature, which often utilizes LLMs on specific types of notes, the methods described herein harnesses RAG and map-reduce to effectively analyze the complete patient documentation. These experiments demonstrated the superiority of this method over SME rule-based models in diagnosing PH. Efficient LLM-based phenotype models offer scalability and improvement in identifying specific diseases in real-world EHRs, reducing the manual workload for SMEs and the need for ad-hoc machine learning models while enabling comprehensive patient record analysis. This advancement promises to enhance systems utilizing EHRs for purposes such as clinical decision support, care gap detection/population health management, clinical trial matching, and cohort generation.

The present disclosure describes, among other things, an AI platform for providing subject discovery, phenotyping, clinical/medical information, and/or subject support. The AI platform may include individual agents that return accurate and relevant information (e.g., identifying target cohorts and/or members of target populations). Each agent may include a language model (optionally trained and/or fine-tuned on a particular domain). The AI platform may also include one or more composite agents that give instructions to, and combines results from, a plurality of task-specific agents configured for different tasks.

The AI platform may include one or more of the following example components. A genetic sequencing component with downstream molecular bioinformatics that operate to call out relevant biomarkers in DNA, RNA, or their derivatives for a specimen that is sequenced and reported back to an ordering physician. A pathology imaging component that operates on cellular/slide level images to identify relevant biomarkers from cells within imaged tissue. A radiological imaging component which operates on larger images of the body through the different radiology imaging technologies to identify the presence or longitudinal progression of tumors in the subject. Each of these components may include, or communicate with, a corresponding agent to identify and/or report information relevant to a user query or request.

As an example, a first agent of the AI platform may receive a user request (e.g., requesting identification of a target population). The first agent may communicate the user request to a second agent (e.g., a retriever component) of the AI platform. For example, the first or second agent may generate a structured call and/or embedding from the user request. The structured call (e.g., an application programming interface (API) call) and/or embedding may be used to retrieve relevant results. The first or second agent may transmit the relevant results to a third agent of the AI platform (e.g., an LLM-based agent), which may identify a subset of the results as responsive to the user request. The first or third agent may reformat the subset of the results and display (or otherwise present) the subset of the results to the user. In some embodiments, an agent is configured for multiple types of tasks. In these embodiments, the agent may identify a user intent (e.g., to identify a target population) and respond accordingly. In some embodiments, an agent is configured for only one type of task (e.g., medical information retrieval or target population membership). In these embodiments, the agent may not identify an intent of the user (e.g., the agent may assume the intent). In some embodiments, the agent receives the intent from a different component of the AI platform or a different system or device. In the above example, each agent may also interface with other agents to obtain additional information related to the user request (such as particular patient records, therapy/drug information, and/or relevant guidelines). In some embodiments, an agent includes a pretrained language model (e.g., trained on a particular domain and/or using particular databases). In some embodiments, an agent queries an unstructured database (e.g., in addition, or alternatively, to generating a structured call).

The AI platform, or components thereof, may be used in conjunction with any medical field (e.g., to assist physicians in the treatment of any associated disease state therein), such as on oncology, endocrinology (e.g., diabetes), mental health (e.g., depression and related pharmacogenetics), and cardiovascular disease. For example, the AI platform may also include a cardiology-based component (agent) that operates on ECG data to identify subjects of high risk for cardiovascular disease. As another example, the AI platform may include a data curation component (agent) that obtains raw (unstructured) data and structures it into a common and useful format as a repository (e.g., a multimodal database) of clinical data from which other agents/models may operate. As another example, the AI platform may search within the clinical data to identify cohorts of related subjects and to generate insights and/or analytics. As another example, the AI platform may monitor an electronic health record (EHR) to identify care gaps and/or reminders to physicians to take action with a respective subject. In this way, the AI platform may serve as a docket manager for physicians and identifies issues/events the physicians did not manually docket to ensure patients get the timely care they need. The AI platform may also track and/or catalog relevant therapies (e.g., on label and/or off label use) for a set of disease states. The AI platform may also track and/or catalog relevant clinical trials (e.g., in multiple countries and/or from multiple authorities) for a set of disease states.

As discussed below, the AI platform may include an AI-enabled clinical assistant that provides access to patient insights. The AI-enabled clinical assistant may use one or more language models and/or other types of generative AI. The AI platform may also include a hub component that allows physicians to order, track, and view test results, export patient data, and provides insights into genomic alterations, treatment implications and clinical trial matching. The hub component may be used in conjunction with the AI-enabled clinical assistant to allow physicians to interact using conversational language including natural language inputs and follow-up questions and remarks. The AI platform may also include a peer-to-peer messaging component for physicians and other medical experts to share knowledge, insight, and/or perspective on medical fields such as molecular oncology (e.g., as it pertains to patient care). The messaging component may be used in conjunction with the AI-enabled clinical assistant to engage in, and optionally learn from, the conversations on the messaging component. For example, the AI-enabled clinical assistant may be invoked in conversation to provide insights and/or data for a particular topic or conversation. The AI platform may also include an EHR interface component configured to allow physicians, and optionally other users, to view, edit, and/search an EHR. The EHR interface component may be communicatively coupled with one or more services and/or databases to obtain updated information and reports (e.g., via push notifications). The EHR interface component may be used in conjunction with the AI-enabled clinical assistant to search, edit, summarize, and/or reform an EHR. The AI platform may also include a research analytical component that provides de-identified patient/clinical data and insights.

Reference will now be made to embodiments, examples of which are illustrated in the accompanying drawings. In the following description, numerous specific details are set forth in order to provide an understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

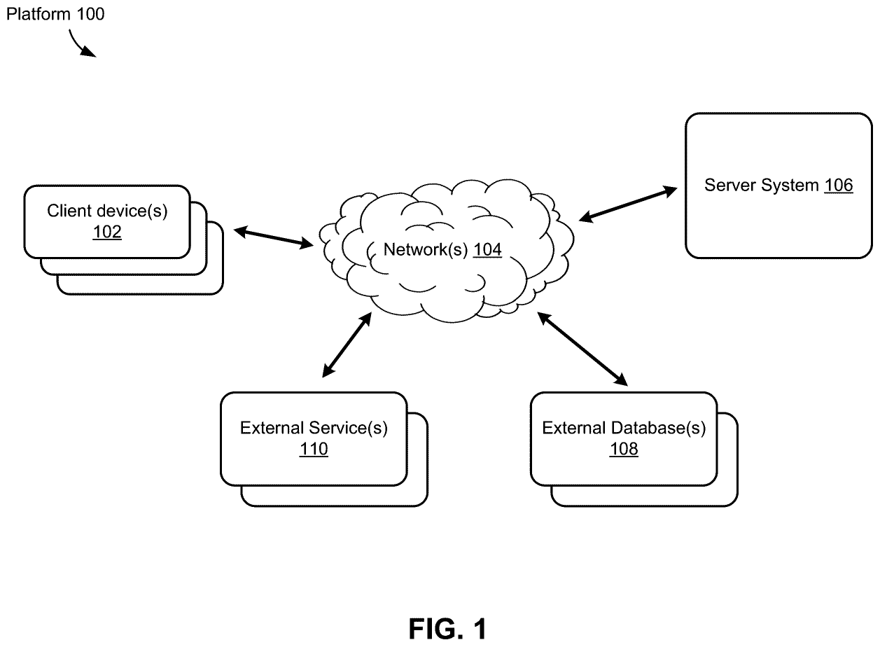

FIG. 1 is a block diagram illustrating a platform 100 in accordance with some embodiments. In some embodiments, the platform 100 is an AI platform (e.g., the AI platform discussed previously). The platform 100 includes one or more client devices 102 communicatively coupled to a server system 106 via one or more networks 104. In accordance with some embodiments, the platform 100 further includes, or communicates with, one or more external services 110 and one or more external databases 108. In some embodiments, the one or more networks 104 include public communication networks, private communication networks, or a combination of both public and private communication networks. For example, the one or more networks 104 can be any network (or combination of networks) such as the Internet, other wide area networks (WAN), local area networks (LAN), virtual private networks (VPN), metropolitan area networks (MAN), peer-to-peer networks, and/or ad-hoc connections. In some embodiments, the platform 100 includes only a subset of the components shown in FIG. 1. For example, the platform 100 may include only one of: of a client device 102 or a server system 106.

In some embodiments, a client device 102 is associated with one or more users. In some embodiments, a client device 102 is a personal computer, mobile electronic device, wearable computing device, laptop computer, tablet computer, mobile phone, feature phone, smart phone, a speaker, television (TV), and/or any other electronic device capable of interacting with a user (e.g., an electronic device having an I/O interface). The client device(s) 102 may communicatively couple to other components of the platform 100 wirelessly and/or through a wired connection (e.g., directly through an interface, such as an HDMI interface).

In some embodiments, the client device(s) 102 send and receive information, such as queries and results, through network(s) 104. For example, the client device(s) 102 may send a query or request to the server system 106, the external service(s) 110, and/or the external database(s) 108 through network(s) 104. As another example, the client device(s) 102 may receive results and other responses from the server system 106, the external service(s) 110, and/or the external database(s) 108 through network(s) 104. In some embodiments, two or more client devices 102 communicate with one another (e.g., resending and responding to queries and requests). The two or more client devices 102 may communicate via the network(s) 104 or directly (e.g., via a wired connection or through a peer-to-peer wireless connection).

In some embodiments, the server system 106 includes multiple electronic devices communicatively coupled to one another. In some embodiments, the multiple electronic devices are collocated (e.g., in a datacenter), while in other embodiments, the multiple electronic devices are geographically separated from one another. In some embodiments, the server system 106 stores and provides clinical and/or patient data. In some embodiments, the server system 106 trains, publishes, and/or utilities one or more agents and/or language models. In some embodiments, the server system 106 receives and responds to queries and requests from the client device(s) 102 using the one or more agents and/or language models. In some embodiments, the server system 106 includes multiple nodes and/or clusters configured to handle different types of tasks and/or handle requests and queries from different geographical locations.

In some embodiments, the client device(s) 102 and/or the server system 106 communicate with the external service(s) 110 and/or the external database(s) 108 via an application programming interface (API). In some embodiments, the external service(s) 110 and/or the external database(s) 108 are maintained/operated by a third party to the platform 100. In some embodiments, the external service(s) 110 include agents, location services, time services, web-enabled services, and/or services that access information stored external to the platform 100.

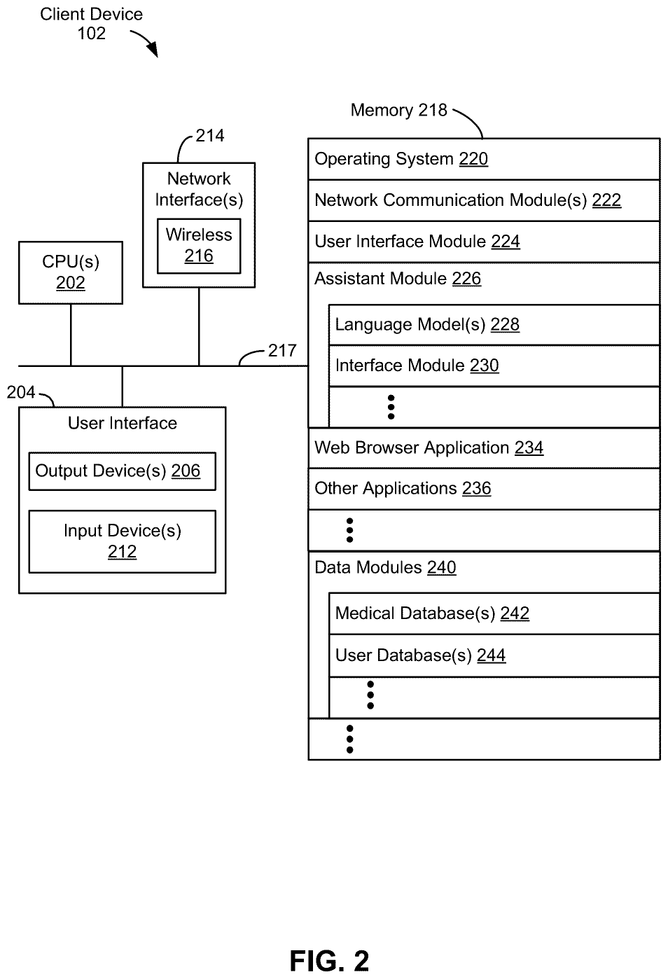

FIG. 2 is a block diagram illustrating a client device 102 in accordance with some embodiments. The client device 102 includes one or more central processing units (CPUs) 202, a user interface 204, one or more network (or other communications) interfaces 214, memory 218, and one or more communication buses 217 for interconnecting these components. In some embodiments, the client device 102 includes a processor or other control circuitry (e.g., in addition, or alternatively, to the CPUs 202). The communication buses 217 optionally include circuitry (sometimes called a chipset) that interconnects and controls communications between system components. Optionally, the client device 102 includes a location-detection component, such as a global navigation satellite system (GNSS) (e.g., GPS (global positioning system), GLONASS, Galileo, BeiDou) or other geo-location receiver, and/or location-detection software for determining the location of the client device 102.

In some embodiments, client device 102 includes one or more sensors including, but not limited to, accelerometers, gyroscopes, compasses, magnetometer, light sensors, near field communication transceivers, barometers, humidity sensors, temperature sensors, proximity sensors, range finders, and/or other sensors/devices for sensing and measuring various environmental conditions.

The user interface 204 includes output device(s) 206 and input device(s) 212. In some embodiments, the input device(s) 212 include a keyboard, mouse, a track pad, and/or a touchscreen. In some embodiments, the user interface 204 includes a display device that includes a touch-sensitive surface, in which case the display device is a touch-sensitive display. In client devices that have a touch-sensitive display, a physical keyboard is optional (e.g., a soft keyboard may be displayed when keyboard entry is needed). In some embodiments, the output device(s) 206 include a speaker and/or a connection port for connecting to speakers, earphones, headphones, or other external listening devices. In some embodiments, the input device(s) 212 include a microphone and/or voice recognition device to capture audio (e.g., speech from a user).

In some embodiments, the one or more network interfaces 214 include wireless and/or wired interfaces for receiving data from and/or transmitting data to other client devices 102, the server system 106, and/or other devices or systems. The data communications may be carried out using any of a variety of custom or standard wireless protocols (e.g., NFC, RFID, IEEE 802.15.4, Wi-Fi, ZigBee, 6LoWPAN, Thread, Z-Wave, Bluetooth, ISA100.11a, WirelessHART, MiWi, etc.). Furthermore, the data communications may be carried out using any of a variety of custom or standard wired protocols (e.g., USB, Firewire, Ethernet, etc.). For example, the one or more network interfaces 214 may include a wireless interface 216 for enabling wireless data communications with other client devices 102, systems, and/or or other wireless (e.g., Bluetooth-compatible) devices. Furthermore, in some embodiments, the wireless interface 216 (or a different communications interface of the one or more network interfaces 214) enables data communications with other WLAN-compatible devices and/or the server system 106 (via the one or more network(s) 104).

The memory 218 includes high-speed random-access memory, such as DRAM, SRAM, DDR RAM, or other random-access solid-state memory devices; and may include non-volatile memory, such as one or more magnetic disk storage devices, optical disk storage devices, flash memory devices, or other non-volatile solid-state storage devices. The memory 218 optionally includes one or more storage devices remotely located from the CPU(s) 202. The memory 218, or alternately, the non-volatile memory solid-state storage devices within the memory 218, includes a non-transitory computer-readable storage medium. In some embodiments, the memory 218 or the non-transitory computer-readable storage medium of the memory 218 stores the following programs, modules, and data structures, or a subset or superset thereof:
an operating system 220 that includes procedures for handling various basic system services and for performing hardware-dependent tasks;
network communication module(s) 222 for connecting the client device 102 to other computing devices connected to one or more network(s) 104 via the one or more network interface(s) 214 (wired or wireless);
a user interface module 224 that receives commands and/or inputs from a user via the user interface 204 (e.g., from the input device(s) 212) and provides outputs via the user interface 204 (e.g., the output device(s) 206);
an assistant module 226 that engages with a user in an integrated, conversational manner using natural language dialog, and invokes external services when appropriate to obtain information or perform various actions. In some embodiments, the assistant module 226 works in conjunction with an assistant module at the server system 106 (e.g., the assistant module 316). In some embodiments, the assistant module 226 includes the following modules (or sets of instructions), or a subset or superset thereof:
   one or more language models 228 that engage with a user and/or perform specific tasks in furtherance of a user request or query. In some embodiments, the language model(s) 228 include one or more large language models, such as GPT-3, GPT-4, BioGPT, and PaLM-2; and
   an interface module 230 that allows the language model(s) 228 communicate with other applications, components, and devices (e.g., via an API or structured query). In some embodiments, the interface module 230 is, or includes, an agent discussed herein;
a web browser application 234 for accessing, viewing, and interacting with web sites;
other applications 236, such as applications for word processing, calendaring, mapping, weather, stocks, time keeping, virtual digital assistant, presenting, number crunching (spreadsheets), drawing, instant messaging, e-mail, telephony, video conferencing, photo management, video management, a digital music player, a digital video player, 2D gaming, 3D (e.g., virtual reality) gaming, electronic book reader, and/or workout support; and
one or more data modules 240 for handling the storage of and/or access to data such as medical data, clinical data, patient data, and user data. In some embodiments, the one or more data modules 240 include:
   one or more medical databases 242 for storing medical data (e.g., regarding therapies, drugs, treatments, patients, cohorts and/or diseases); and
   one or more user databases 244 for storing user data such as user preferences, user settings, and other metadata.

In some embodiments, the memory 218 includes one or more modules not shown in FIG. 2. For example, the memory 218 may include one or more agent modules (e.g., a retriever component) that are distinct from the assistant module 226. In some embodiments, the client device 102 includes one or more standalone agents (e.g., that execute and operate at the client device 102) and/or one or more dependent agents (e.g., that operate in conjunction with a component at a remote device, such as the server system 106). In some embodiments, one or more agents are generated/trained at the server system 106 and deployed at the client device 102.

Although FIG. 2 illustrates the client device 102 in accordance with some embodiments, FIG. 2 is intended more as a functional description of the various features that may be present in a client device than as a structural schematic of the embodiments described herein. In practice, and as recognized by those of ordinary skill in the art, items shown separately could be combined and some items could be separated.

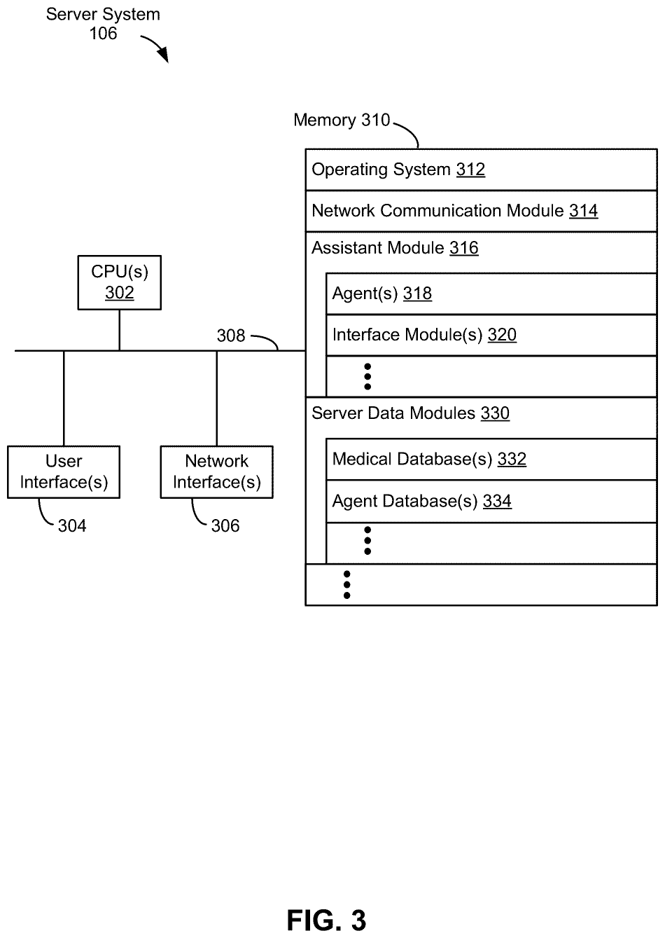

FIG. 3 is a block diagram illustrating a server system 106 in accordance with some embodiments. In accordance with some embodiments, the server system 106 includes one or more CPUs 302, one or more user interfaces 304, one or more network interfaces 306, memory 310, and one or more communication buses 308 for interconnecting these components.

The memory 310 includes high-speed random-access memory, such as DRAM, SRAM, DDR RAM, or other random access solid-state memory devices; and may include non-volatile memory, such as one or more magnetic disk storage devices, optical disk storage devices, flash memory devices, or other non-volatile solid-state storage devices. The memory 310 optionally includes one or more storage devices remotely located from one or more CPUs 302. The memory 310, or, alternatively, the non-volatile solid-state memory device(s) within the memory 310, includes a non-transitory computer-readable storage medium. In some embodiments, the memory 310, or the non-transitory computer-readable storage medium of the memory 310, stores the following programs, modules and data structures, or a subset or superset thereof:
an operating system 312 that includes procedures for handling various basic system services and for performing hardware-dependent tasks;
a network communication module 314 that is used for connecting the server system 106 to other computing devices connected to one or more networks 104 via one or more network interfaces 306 (wired or wireless);
an assistant module 316 that engages with a user (e.g., a remote user) in an integrated, conversational manner using natural language dialog, and invokes external services when appropriate to obtain information or perform various actions. In some embodiments, the assistant module 316 works in conjunction with an assistant module at a client device 102 (e.g., the assistant module 226). In some embodiments, the assistant module 316 includes the following modules (or sets of instructions), or a subset or superset thereof:
   one or more agents 318 that are configured to perform specific tasks or perform tasks within specific domains (e.g., any of the agents described herein, such as a retriever agent and a target population membership agent); and
   one or more interface modules 320 that allows the agent(s) 318 to communicate with other agents, applications, components, and devices (e.g., via an API or structured query); and
one or more server data modules 330 for handling the storage of and/or access to data (e.g., clinical and user data). In some embodiments, the one or more server data modules 330 include:
   one or more medical databases 332 for storing medical data (e.g., regarding therapies, drugs, treatments, patients, cohorts, and/or diseases); and
   one or more agent databases 334 for storing agent data such as settings, training, instructions, and other metadata.

In some embodiments, the server system 106 includes web or Hypertext Transfer Protocol (HTTP) servers, File Transfer Protocol (FTP) servers, as well as web pages and applications implemented using Common Gateway Interface (CGI) script, PHP Hyper-text Preprocessor (PHP), Active Server Pages (ASP), Hyper Text Markup Language (HTML), Extensible Markup Language (XML), Java, JavaScript, Asynchronous Javascript and XML (AJAX), XHP, Javelin, Wireless Universal Resource File (WURFL), and the like.

In some embodiments, the memory 310 includes one or more modules not shown in FIG. 3. For example, the memory 310 may include one or more agent modules (e.g., a retriever component) that are distinct from the assistant module 316. In some embodiments, the server system 106 includes one or more standalone agents (e.g., that execute and operate at the server system 106) and/or one or more dependent agents (e.g., that operate in conjunction with a component at a remote device, such as a client device 102).

Although FIG. 3 illustrates the server system 106 in accordance with some embodiments, FIG. 3 is intended more as a functional description of the various features that may be present in a server system than as a structural schematic of the embodiments described herein. In practice, and as recognized by those of ordinary skill in the art, items shown separately could be combined and some items could be separated. For example, some items shown separately in FIG. 3 could be implemented on single servers and single items could be implemented by one or more servers. In some embodiments, the clinical database(s) and/or the agent database(s) 334 are stored on devices that are accessed by the server system 106 (e.g., the external database(s) 108). The actual number of servers used to implement the server system 106, and how features are allocated among them, will vary from one implementation to another and, optionally, depends in part on an amount of data traffic that the server system handles during peak usage periods as well as during average usage periods.

Each of the above identified modules stored in the memory 218 and 310 corresponds to a set of instructions for performing a function described herein. The above identified modules or programs (e.g., sets of instructions) need not be implemented as separate software programs, procedures, or modules, and thus various subsets of these modules may be combined or otherwise re-arranged in various embodiments. In some embodiments, the memory 218 and 310 optionally store a subset or superset of the respective modules and data structures identified above. Furthermore, the memory 218 and 310 optionally store additional modules and data structures not described above.

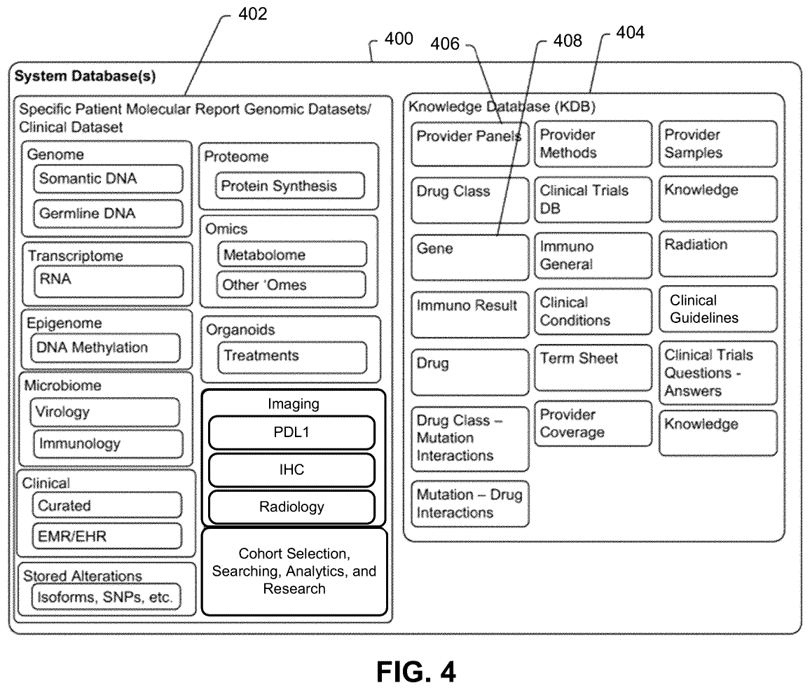

FIG. 4 is a block diagram illustrating system database(s) 400 in accordance with some embodiments. At least a portion of the system database(s) 400 is optionally stored at a client device 102 (e.g., as the medical database(s) 242), the server system 106 (e.g., as the medical database(s) 332), and/or the external database(s) 108. The system database 400 includes molecular report genomic datasets and clinical datasets 402 and a non-patient specific knowledge database (KDB) 404 in accordance with some embodiments. The datasets 402 include, among other data, genome, transcriptome, epigenome, microbiome, clinical, stored alterations proteome,-omics, organoids, imaging and cohort and propensity data sets which are described in other patent applications in some detail. For example, the cohort selection, searching, analytics, and research datasets may include data about tumors of unknown origin (TUO) predictors, metastasis predictors, and survival analytics. As an example, the imaging datasets may include radiology imaging data, immunohistochemistry imaging data, positron emission tomography (PET) data, and/or single-photon emission computed tomography (SPECT) imaging data. The imaging datasets my include data regarding nodule identifiers, tracking, and/or longitudinal analytics. The KDB 404 includes separate sub-databases related to specific information types including, as shown, provider panels 406 (e.g., information related to genetic panels supported by the service provider that operates the system), drug classes (e.g., drug class specific information (e.g., do drugs of a specific class work on pancreatic cancer, what drugs are considered to be included in a specific drug class, etc.)), specific genes 408, immuno results (e.g., information related to treatments based on specific immuno biomarker results), specific drugs, drug class-mutation interactions, mutation-drug interactions, provider methods (e.g., questions about processes performed by the service provider), clinical trials, immuno general, clinical conditions such as clinical diseases, term sheets (e.g., definitions of industry specific terms), provider coverage (e.g., information about provider tests and results), provider samples (e.g., information about types of samples that can be processed by the provider), knowledge (e.g., scripted questions and answers on various frequently asked questions that do not fall into other sub-databases), radiation (e.g., information related to suitable radiation treatments given specific cancer states), clinical guidelines (e.g., national guidelines related to classification of cancer states, accepted treatments, etc.) and clinical trials questions-answers (e.g., information related to locations and administrators of clinical trials. Organizing the KDB 404 into sub-databases may make it easier to manage those databases as information therein evolves over time and also enables addition of new sub-databases related to other defined information types. In some embodiments, the clinical datasets 402 and/or the KDB 404 is arranged in a different manner than is shown in FIG. 4 (e.g., with different sub-databases and/or with a different organizational scheme).

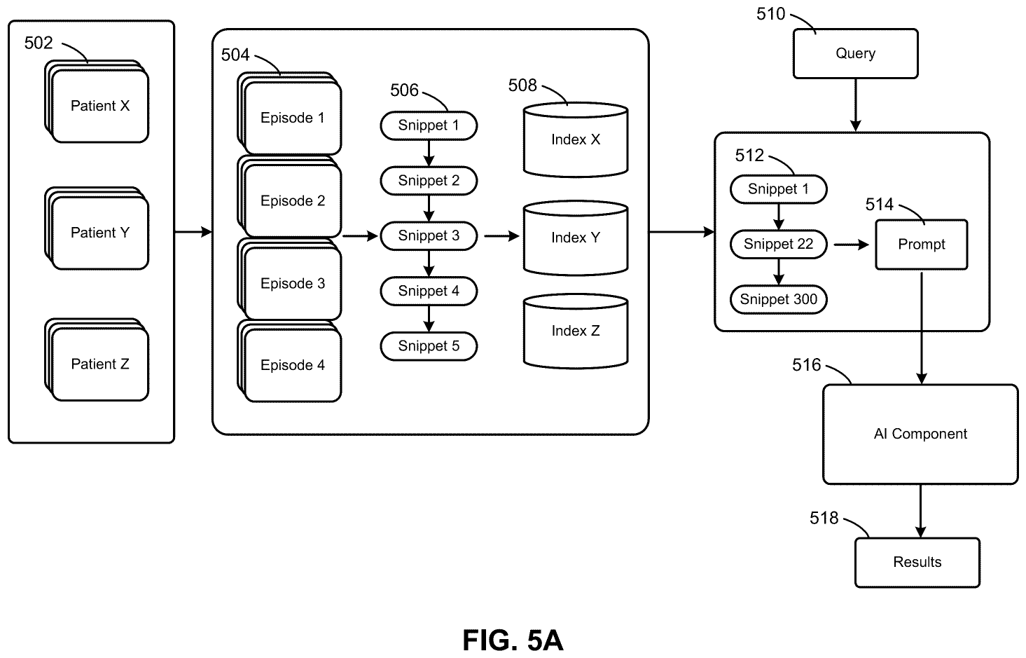

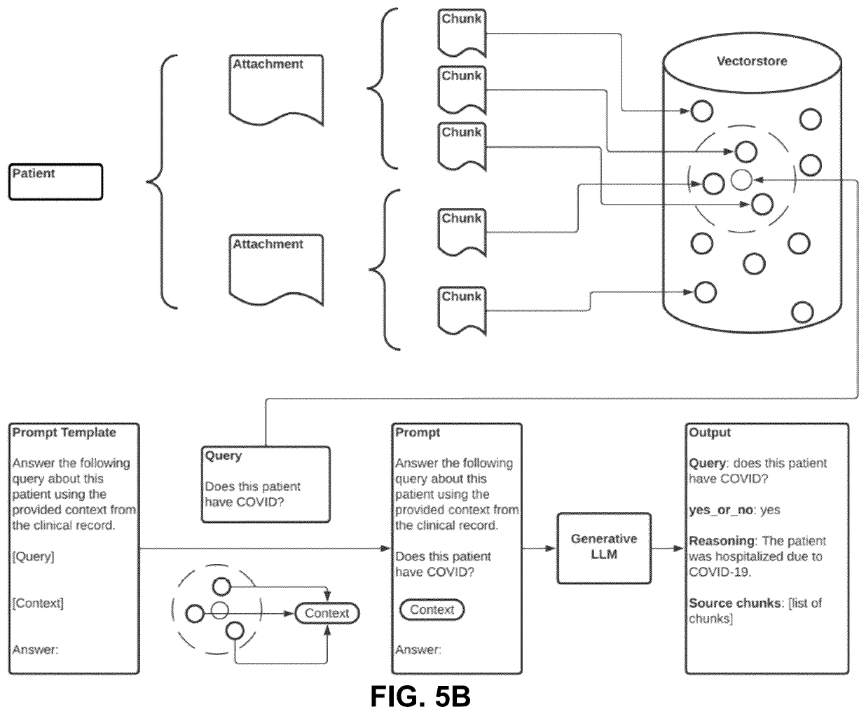

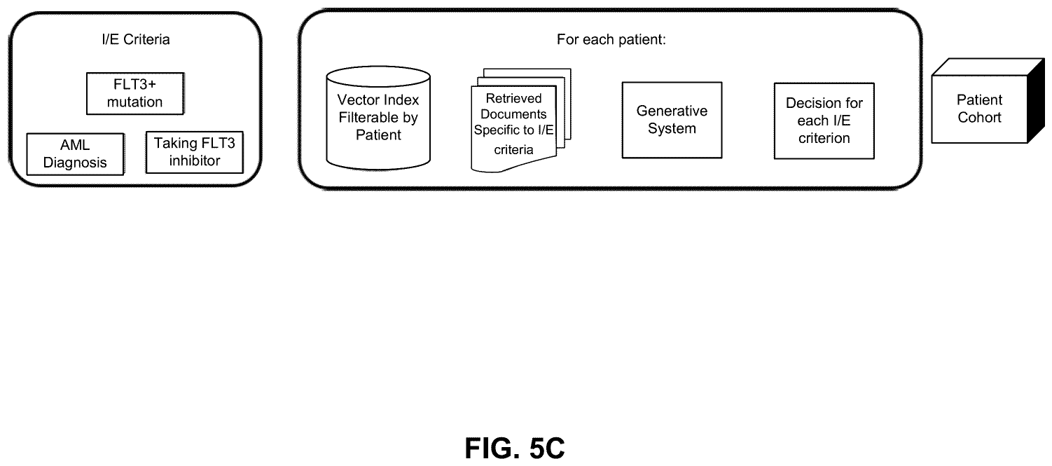

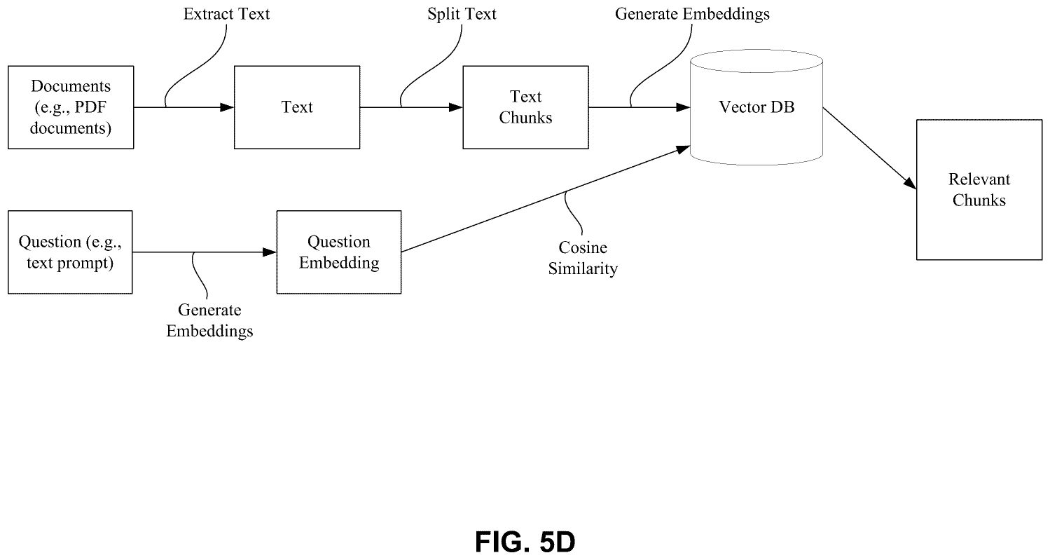

FIGS. 5A-5D are block diagrams illustrating example patient discovery processes in accordance with some embodiments. As shown in FIG. 5A, patient data 502 (e.g., medical data such as EHRs) is obtained for a set of patients (e.g., patients X, Y, and Z). The patient data 502 may include one or more charts, records, and/or attachments for each patient. The patient data 502 may be obtained from one or more medical databases (e.g., medical database(s) 242 and/or medical database(s) 332). The one or more medical databases may include two databases having different formats, structures, and/or types of data. In some embodiments, the set of patients is 10 or more, 100 or more, 500 or more, 1000 or more, 10,000 or more or 100,000 or more patients. In some embodiments, the set of patients is between 10 and 100,000 patients, between 100 and 1×106 patients, between 500 and 5×106 patients, between 1000 and 2 million patients, or between 10,000 and 5 million patients.

In accordance with some embodiments, portions of the patient data 502 are prepared and/or stored for subsequent querying. For example, patient files are chunked and indexed in a manner that makes them easy to search by a retriever component. In such chunking, chunks of consecutive text are extracted from each patient file and stored as chunks. In some embodiments, each chunk consists of between 100 and 1000 characters. In typical embodiments the chunking algorithm makes the chunks overlapping. For example, in one chunking algorithm in accordance with the present disclosure, the chunks each consist of 512 characters and each chunk has 128 characters of overlap with another chunk extracted from the medical record of a patient. In this way, relevant patient snippets (where the term snippet and chunk is used interchangeably herein) along with their metadata (e.g., which medical record they came from) can be searched and retrieved for an LLM to use as context. These snippets may include transforms such as embeddings to make search easier. For instance, the chunks may be embedded into numerical vectors using known techniques for conversion of chunks of ASCII text to numerical vector format. As shown in FIG. 5A, portions of the patient data 502 are curated into episodes 504 (e.g., sets of documents). Snippets 506 are obtained from the curated episodes 504 (e.g., in a linked list). For example, each document may be split into 8-12 snippets. In some embodiments, the snippets 506 are obtained from the curated episodes 504 via a chunking process. In some embodiments, the snippets 506 are time ordered. For instance, in some embodiments, a time stamp of creation of each document of each medical record of a patient is propagated as metadata to the snippets created from such documents and used to time order the snippets. The snippets 506 are indexed into one or more datasets 508. In some embodiments, the snippets 506 are reformatted, structured, and/or vectorized prior to being stored in the datasets 508. In some embodiments, an embedding is generated for each snippet 506 and the embeddings are stored in the datasets 508 (and/or stored in an embedding space). In some embodiments, each snippet is restricted to 512 characters in length (e.g., with 128-character overlap).

A query 510 is obtained (e.g., is obtained via a digital assistant or graphical user interface described herein) and snippets 512 relevant to the query 510 are retrieved from the datasets 508. In one example, the query 510 is a request to identify a target population having one or more predefined characteristics. In some embodiments, a predetermined number of snippets are retrieved. In some embodiments, snippets having at least a predetermined similarity score are retrieved. In some embodiments, the snippets are retrieved using a retriever component (e.g., a task-specific agent). For example, the retriever component is configured to search one or more patient indices to find data relevant to a particular task. In some embodiments, the retriever component uses regular expressions, sparse vector searches, and/or dense vector searches to retrieve the relevant snippets. In some embodiments, the top k results are obtained and optionally ranked by the retriever component.

The relevant snippets 512 are incorporated into a prompt 514 for an AI component 516. In some embodiments, the relevant snippets 512 are combined with one or more prompt instructions in a single prompt. In some embodiments, the relevant snippets 512 are provided to the AI component 516 in two or more prompts (e.g., a sequence of prompts). In some embodiments, the one or more prompt instructions instruct the AI component how to analyze the relevant snippets 512. In some embodiments, the prompt 514 includes relevant patient information from the retriever model and instructs the LLM in how to decide on patient categorization. In some embodiments, the prompt 514 contains intermediate information, such as the LLM's reasoning and/or previous answers. In some embodiments, the relevant snippets 512 correspond to inclusion and/or exclusion criteria for a target population (e.g., must have BRCA1 germline mutation). Example inclusion criteria include “must have covid-19”, “must have NSCLC”, and “must be on platinum-based chemotherapy, must have received fulvestrant monotherapy as secondary or tertiary LoT after CDK4/6i+ET in metastatic setting.” An example prompt is “Use the following pieces of context to answer the multiple-choice question at the end. Answer the question as one of [” Yes “, “No”, or “Inconclusive evidence”]. Do not add further explanation.”

The AI component 516 provides results 518 responsive to the prompt 514. For example, the AI component 516 may identify members of a target population by analyzing the relevant snippets 512. In some embodiments, the results are transmitted to and/or provided at a client device 102 (e.g., displayed in a user interface). In some embodiments, the results 518 are stored in a table or dataset. In some embodiments, the results 518 include identifiers for one or more patients. In some embodiments, the results 518 include contact information for the one or more patients. As an example, the query 510 may ask whether a particular patient is a member of a target population and the results 518 may include an answer (e.g., yes or no), a rationale, a confidence score, and/or a basis for the answer.

In some embodiments, the patient data 502 is anonymized to ensure privacy for the patients. In some embodiments, the results 518 include statistics and evaluation of the patient data 502. For example, the AI component 516 may identify a number of patients that may be members of a particular target population.

FIG. 5B illustrates a process of obtaining attachments (e.g., documents and/or other medical information) for a patient and storing chunk embeddings in a vector store to use as context for subsequent queries. In some embodiments, the attachments include one or more PDF documents, and the chunking process includes performing optical character recognition on the PDF documents to obtain the chunks. In some embodiments, each attachment is provided to an AI component (e.g., an LLM) without undergoing the chunking process (e.g., the AI component is configured to input a whole attachment). In some embodiments, a tuple is generated from a user query (e.g., the tuple includes patient and inclusion/exclusion criteria) and the language model generates a decision (e.g., a yes/no decision) based on the tuple. FIG. 5C illustrates example inclusion/exclusion criteria and an example process for determining whether each patient meets the inclusion/exclusion criteria.

An example of configuration parameters for a process for finding patients that meet inclusion/exclusion criteria for a cohort (e.g., as shown in FIGS. 5A and 5B) is shown below in Example 1.

{
‘cache_path’:
‘cohort_name’:
‘n_patients’:
‘embedding_batch_size’:
‘device’:
‘doc_chunk_size’:
‘doc_chunk_overlap’:
‘text_seperators’:
‘embedding_model’:
‘instructor_embed_instruction’:
‘instructor_query_instruction’:
‘prompt_template_name’:
‘generator_queries’:
‘vector_store_db_name’:
‘generator_model’:
‘n_retrieved_docs’:
‘temperature’:
‘top_p’:
‘tok_k’:
‘max_output_tokens’:
‘output_parser’:
‘chunking_config_hash’:
‘embedding_config_hash’:
}

### Example 1—Example Configuration Parameters

In some embodiments, the documents are split into chunks and/or snippets based on fixed character length (with optional overlap), fixed token length (with optional overlap), and/or section-based splitting (e.g., identifying section headings and splitting on those). In some embodiments, a prompt for the AI component (e.g., the prompt 514) includes retrieved patient context, inclusion/exclusion criteria, and a question to determine if the patient satisfies the criteria.

FIG. 5D shows a process of generating embeddings from documents (e.g., PDF documents). In some embodiments, the documents are obtained from a medical database (e.g., clinical trial protocol documents form a clinical trial database). In some embodiments, the medical database is a plurality of medical databases. In some embodiments, the medical documents of more than 10, more than 100, more than 1000, more than 10,000 or more than 100,000 patients are extracted, split into chunks and embedded into the vector database. In some embodiments, there are more than 2, more than 3, more than 4, more than 10, more than 20 documents associated with each patient for which text is extracted, split into chunks and embedded as vector in the vector database. In some embodiments, each chunk includes metadata that indicates the source subject in the medical database from which the chunk originated. In some embodiments, each embedding includes metadata that indicates the source subject in the medical database from which the embedding originated. In some embodiments, for each respective patient for which text is extracted, there is more than 100 kilobytes of text data across the documents associated with the respective patient that is extracted. In some embodiments, for each respective patient for which text is extracted, there is more than a megabyte of text data across the documents associated with the respective patient that is extracted. In some embodiments, for each respective patient for which text is extracted, there is more than a gigabyte of text data across the documents associated with the respective patient that is extracted. In some embodiments, embeddings are also generated from metadata corresponding to the documents and stored in the vector database.

FIG. 5D also shows a process of generating a question embedding and using a similarity between the question embedding and vectors in a vector database to identify relevant chunks of the documents. In some embodiments, the vector similarity is based on a cosine distance, Euclidian distance, Manhattan distance, Jaccard distance, correlation distance, Chi-square distance, Mahalanobis distance, and/or a semantic comparison of embeddings. Consider X^{p}=[X₁^{p}, . . . , Xₙ^{p}] and X^{q}=[X₁^{p}, . . . , Xₙ¹] to be two embeddings to be compared (the question embedding and a vector in the vector database). Also consider maxi and min; to be the maximum value and the minimum value of an i^{th} attribute of a vector in the vector database, respectively. The distance between X^{p} and X^{q} is defined as follows for each distance metric:

| Type | Distance Metric |
|---|---|
| Euclidean | $d ( {X}^{p} , {X}^{q} ) = \sqrt{{\sum }_{i = 1}^{n} {( {X}_{i}^{p} - {X}_{i}^{q} )}^{2}}$ |
| Manhattan | $d ( {X}^{p} , {X}^{q} ) = {\sum }_{i = 1}^{n} \vert {X}_{i}^{p} - {X}_{i}^{q} \vert$ |
| Maximum | d(X^{p}, X^{q}) = argmaxᵢ \|Xᵢ^{p} − Xᵢ^{q}\| |
| Value | |
| Normalized Euclidean | $d ( {X}^{p} , {X}^{q} ) = \sqrt{\frac{1}{n} {\sum }_{i = 1}^{n} {( \frac{{X}_{i}^{p} - {X}_{i}^{q}}{{max}_{i} - {min}_{i}} )}^{2}}$ |
| Normalized Manhattan | $d ( {X}^{p} , {X}^{q} ) = \frac{1}{n} {\sum }_{i = 1}^{n} \frac{\vert {X}_{i}^{p} - {X}_{i}^{q} \vert}{{max}_{i} - {min}_{i}}$ |
| Normalized Maximum Value | $d ( {X}^{p} , {X}^{q} ) = arg {max}_{i} \frac{\vert {X}_{i}^{p} - {X}_{i}^{q} \vert}{{max}_{i} - {min}_{i}}$ |
| Dice Coefficient | $d ( {X}^{p} , {X}^{q} ) = 1 - \frac{2 {\sum }_{i - 1}^{n} {X}_{i}^{p} {X}_{i}^{q}}{{\sum }_{i - 1}^{n} {X}_{i}^{{p}^{2}} + {\sum }_{i - 1}^{n} {X}_{i}^{{q}^{2}}}$ |
| Cosine coefficient | $d ( {X}^{p} , {X}^{q} ) = 1 - \frac{{\sum }_{i - 1}^{n} {X}_{i}^{p} {X}_{i}^{q}}{\sqrt{{\sum }_{i - 1}^{n} {X}_{i}^{{p}^{2}} \cdot {\sum }_{i - 1}^{n} {X}_{i}^{{q}^{2}}}}$ |
| Jaccard coefficient | $d ( {X}^{p} , {X}^{q} ) = 1 - \frac{{\sum }_{i - 1}^{n} {X}_{i}^{p} {X}_{i}^{q}}{{\sum }_{i - 1}^{n} {X}_{i}^{{p}^{2}} + {\sum }_{i - 1}^{n} {X}_{i}^{{q}^{2}} - {\sum }_{i - 1}^{n} {X}_{i}^{p} {X}_{i}^{q}}$ |

In some embodiments, the question and relevant chunks are input to a large language model (LLM) and the LLM generates an answer (e.g., the relevant chunks are used as context by the LLM for answering the question.

In some embodiments the patient identity of the chunks that are determined to be close in distance to the question embedding is used to select a subject of the patients used to build the vector database. For example, patients having chunks landing in the top-k chunks closest in distance to the question embedding may be culled as a subset of patients that are more fully analyzed by the LLM to determine if they have one or more target characteristics needed for a particular cohort. In one example, a cohort of 500 patients is desired. In this example, the patients are ranked by their best ranked chunk to the question embedding. The top 1000 patients ranked in this manner are then evaluated by the LLM. In some embodiments, the LLM evaluates all the chunks of a selected subject, not just the chunks that were found to be relevant.

In other embodiments, only the most relevant chunks are evaluated by the LLM. For example, the top 10,000, 100,000 or 1×10⁶ chunks in terms of closest distance to the question embedding may be passed on to the LLM for further evaluation.

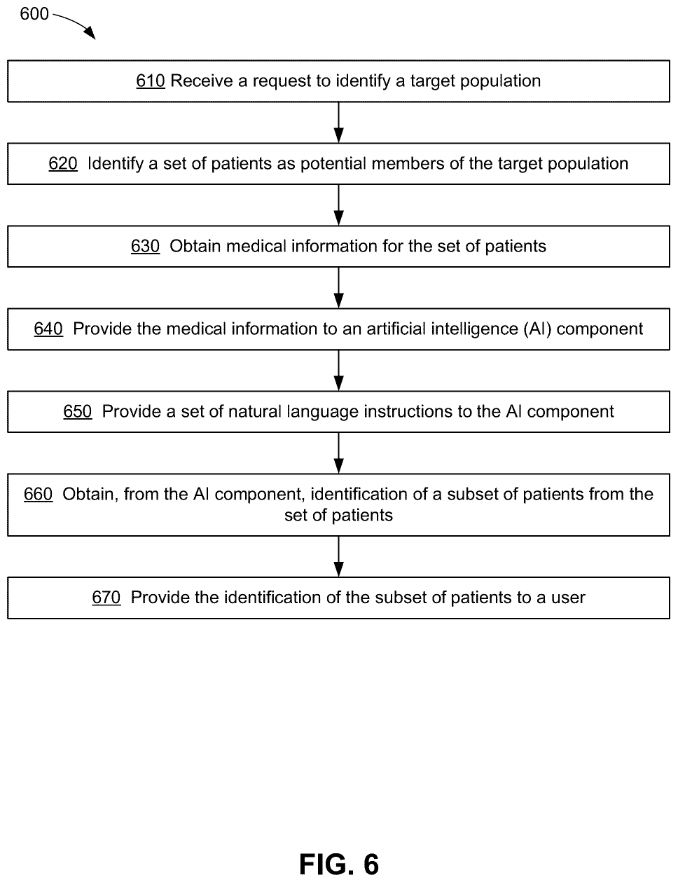

FIG. 6 is a flow diagram illustrating a method 600 of phenotyping in accordance with some embodiments. The method 600 is performed at a computing system (e.g., a client device, server system, and/or service platform) having one or more processors (e.g., the CPUs 202 and/or 302) and memory (e.g., the memory 218 and/or 310). In some embodiments, the memory stores one or more programs configured for execution by the one or more processors. At least some of the operations shown in FIG. 6 correspond to instructions stored in a computer memory or a computer-readable storage medium. In some embodiments, the computing system is the platform 100, the client device(s) 102, and/or the server system 106.

The computing system receives (610) a request to identify a target population. In some embodiments, the request is received from a user (e.g., via an interaction with a digital assistant). In some embodiments, the request is received from a client device (e.g., a client device 102) that is distinct from the computing system.

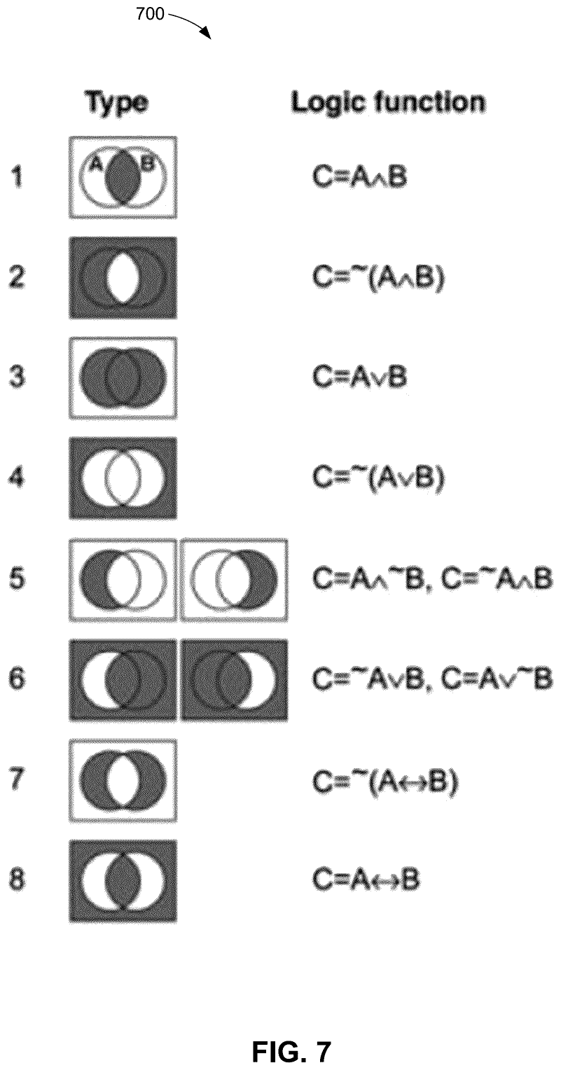

In some embodiments, the request is a request to identify a target population with one or more predefined characteristics. Examples of predefined characteristics include, but are not limited to age, sex, absence of a disease, presence of a disease, stage of a disease, presence of a biomarker (e.g., genetic mutation, etc.), absence of treatment for a condition, history of treatment for a condition, assay result, absence or presence of a tumor, tumor grade, absence or presence of metastasis, etc. In some embodiments the request is one or more logical combinations of such characteristics. In some embodiments, referring briefly to chart 700 of FIG. 7, in some embodiments, the one or more logical operations are Boolean operations. For instance, in some embodiments, a first logical operation describes an “AND” Boolean operation that requires both operands of the logical operation to be satisfied (e.g., the request states that the subject must be female and have cancer. Another example of a logical operation is an “OR” Boolean operation that requires any one condition to be satisfied (e.g., the patient is a cancer patient that has not been treated OR the patient has just recently undergone treatment. Another example of a logical operation is an “EXCLUSIVE OR (XOR)” Boolean operation that requires any one operand of the logical operation to be satisfied and no other element satisfied (e.g., the patient is a male with colon cancer XOR the patient a male with mutation XXX. Other examples of logical operations are a singular “NOT” Boolean operation that requires absence of a characteristic, a plural “NOT” Boolean operation that requires both absence of a first characteristic and presence of a second characteristic. In some embodiments, a logical operation includes a combination of one or more of the above-described logical operations. For instance, in some embodiments, a respective logical operation includes one or more AND, OR, XOR, or NOT operations within the respective logical operation (e.g., an operation including an AND operation and a NOT operation, etc.). However, the present disclosure is not limited thereto.

In some embodiments, the request includes a query regarding whether a particular patient is a member of the target population. In some embodiments, the target population corresponds to a target cohort, exploratory data analysis, and/or consideration of which target best serves downstream training data labeling and/or electrocardiogram (ECG) modeling.

The computing system identifies (620) a set of patients as potential members of the target population. In some embodiments, the computing system uses an agent (e.g., a retriever component) to identify the set of patients. In some embodiments, the set of patients are identified from a patient database and/or a medical database (e.g., the medical databases 242 and/or 332 and/or the external database(s) 108). In some embodiments, a set of patient identifiers are obtained, where the set of patient identifiers correspond to the set of patients. In some embodiments, the set of patient identifiers are anonymized (e.g., correspond to the set of patients, but do not identify the set of patients). In some embodiments, the set of patients are identified based on one or more filters being applied to data in one or more databases. In some embodiments, the set of patients are identified using a logical combination of filters (e.g., reduce the potential universe of patients that are to be reviewed by the AI component). In some embodiments, the filters are combined with one or more logical operation (e.g., logical functions of FIG. 7). In some embodiments, referring briefly to chart 700 of FIG. 7, in some embodiments, one or more logical operations are Boolean operations. For instance, in some embodiments, a first logical operation describes an “AND” Boolean operation that requires two filters to be satisfied. A second logical operation describes an “OR” Boolean operation that requires any one filter to be satisfied. Moreover, a third logical operation describes an “EXCLUSIVE OR (XOR)” Boolean operation that requires any one element (filter) of the third logical operation to be satisfied and no other element satisfied. A fourth logical operation describes a singular “NOT” Boolean operation that requires absence of an element (filter) of the fourth logical operation to be satisfied. A fifth logical operation describes a plural “NOT” Boolean operation that requires both absence of a first element (filter) and presence of a second element (filter) of the fifth logical operation to be satisfied. In some embodiments, a logical operation includes a combination of one or more of the above-described logical operations. For instance, in some embodiments, a respective logical operation includes one or more AND, OR, XOR, or NOT operations within the respective logical operation (e.g., an operation including an AND operation and a NOT operation, etc.). However, the present disclosure is not limited thereto.

As discussed above, in some embodiments, the set of patients are those patients that have vectors in the vector database of FIG. 5D that are close in distance to the embedded query, however the invention is not so limited.

The computing system obtains (630) medical information for the set of patients. In some embodiments, the medical information is obtained from one or more medical databases (e.g., the medical databases 242 and/or 332 and/or the external database(s) 108). In some embodiments, the medical database(s) are owned/operated by third party entities (distinct from the entity that owns/operates the computing system). In some embodiments, the medical database(s) include one or more databases storing structured data and/or one or more databases storing unstructured data. In some embodiments, the medical information includes one or more EHRs and/or patient notes. For example, a retriever model is used to find candidate notes within a patient file.

The computing system provides (640) the medical information to an artificial intelligence (AI) component. In some embodiments, the AI component includes one or more agents. In some embodiments, the AI component includes one or more large language models. In some embodiments, the AI component is a generative AI component. In some embodiments, the medical information is provided to the AI component via one or more prompts. In some embodiments, the medical information is provided to the AI component to provide context for the AI component to process a request/query. In some embodiments, the medical information is in the form of chunks as described above.

The computing system provides (650) a set of natural language instructions to the AI component. In some embodiments, the set of natural language instructions instruct the AI component how to determine if a patient is a member of the target population and/or whether the patient has the one or more predefined characteristics. In some embodiments, the set of natural language instructions are provided to the AI component via one or more prompts. In some embodiments, the computing system provides a set of structured instructions to the AI component.

The computing system obtains (660), from the AI component, identification of a subset of patients from the set of patients. In some embodiments, the identification of the subset of patients includes patient names and/or identifiers. In some embodiments, the AI component provides statistics about the subset of patients in replacement of, or in addition to, providing the identification of the subset of patients.

The computing system provides (670) the identification of the subset of patients to a user. In some embodiments, the computing system sends the identification of the subset of patients (and/or other information from the AI component) to a client device of the user. In some embodiments, the computing system stores the identification of the subset of patients. In some embodiments, the computing system sends statistics about the subset of patients to the user in replace of, or in addition to, the identification of the subset of patients.

Although FIG. 6 illustrates a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. Moreover, it should be recognized that various stages could be implemented in hardware, firmware, software, or any combination thereof.

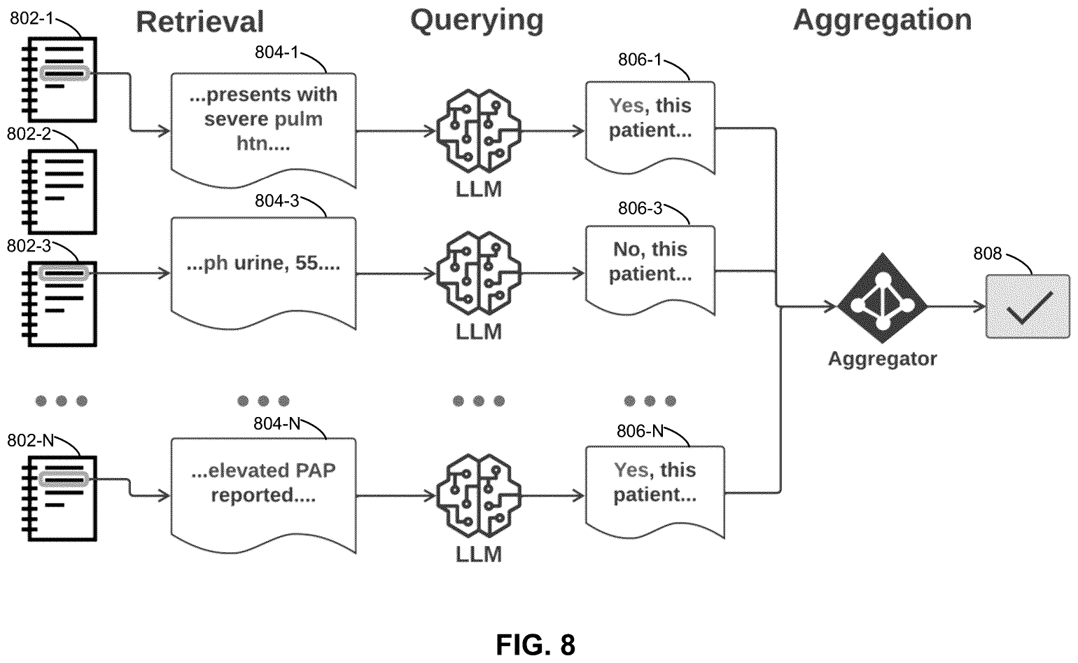

FIG. 8 is a block diagram illustrating an example workflow for an LLM-based phenotyping model, which is based on a retrieval-augmented generation (RAG) approach followed by a map-reduce step for querying and aggregation, in accordance with some embodiments.

Considering the extensive volume of text contained within a real-world data (RWD) warehouse of EHRs, it becomes impractical to process the entirety of a patient's clinical notes within the context window of an LLM. In some embodiments, e.g., as illustrated in FIG. 5, this challenge is addressed by implementing a retrieval-augmented generative (RAG) approach to identify relevant portions of EHR text, e.g., relevant portions of unstructured clinical notes. A RAG approach proves to be more efficient and effective than providing the LLM with larger context windows.

In some embodiments, clinical notes from an EHR are divided into individual segments, also referred to herein as snippets (e.g., snippets 802, as illustrated in FIG. 8). One example method for segmenting unstructured clinical data (e.g., clinical notes) includes tokenizing the unstructured clinical data to obtain a plurality of tokens and segmenting the plurality of tokens to obtain a plurality of segments (snippets), e.g., where each respective segment in the plurality of segments has approximately a same number of tokens.

In some embodiments, the individual snippets are evaluated to determine whether they include information pertinent to determining whether the subject has a target medical condition. In some embodiments, the evaluation is performed by natural language processing. In some embodiments, the evaluation is performed based on pattern recognition of regular expressions (Regex) related to the target medical condition. In some embodiments, the use of Regex avoids introducing bias through additional hyperparameter tuning and narrows the focus to assessing the LLM's capability in diagnosing diseases. However, other retrieval models can be used instead of, or in addition to, Regex. For example, the snippets may be evaluated using Term Frequency-Inverse Document Frequency. In some embodiments, the snippets are evaluated using Cohere's re-rank. In some embodiments, the snippets are evaluated using Instructor embeddings.

In some embodiments, the snippets are retrieved by using a large language model (LLM) to identify portions of a medical record that include information relating to the target medical condition. In some embodiments, a prompt is given to the LLM to identify any portion of a medical record that is relevant to an indication of the disease diagnosis. In some embodiments, the identified portion (e.g., snippet) is defined to be within a specific range of characters. In some embodiments, the identified portion must be from X to Y characters in length, where X is a minimum length and Y is a maximum length. In some embodiments, the identified portion (e.g., snippet) is defined to be within a specific range of token length. In some embodiments, the identified portion must be from X to Y tokens in length, where X is a minimum length and Y is a maximum length. In some embodiments, the identified portion (e.g., snippet) must satisfy a relevance threshold. For instance, in some embodiments, a set of candidate portions are identified and ranked in terms of relevance to the medical condition relative to each other and the top X number of candidate portions are selected for retrieval. In some embodiments, the ranking is limited to portions obtained from a single document within a medical record. In some embodiments, the ranking is applied across a plurality of documents within the medical record.

While the RAG approach reduces the amount of text processed by the LLM, RWD clinical notes often comprise many pages of text. Consequently, the Regex retriever is still likely to return a large number of snippets determined to include information pertinent to determining whether the subject has a target medical condition, which may exceed the LLM's context window. In some embodiments, a map-reduce approach is employed to address this issue. Map-reduce allows for parallel execution of the LLM on individual snippets, improving efficiency and reducing processing time. It also facilitates handling of large numbers of identified snippets by distributing the processing load across multiple iterations. By generating individual outputs for each snippet, the chain can extract specific information that contributes to a more comprehensive final result.

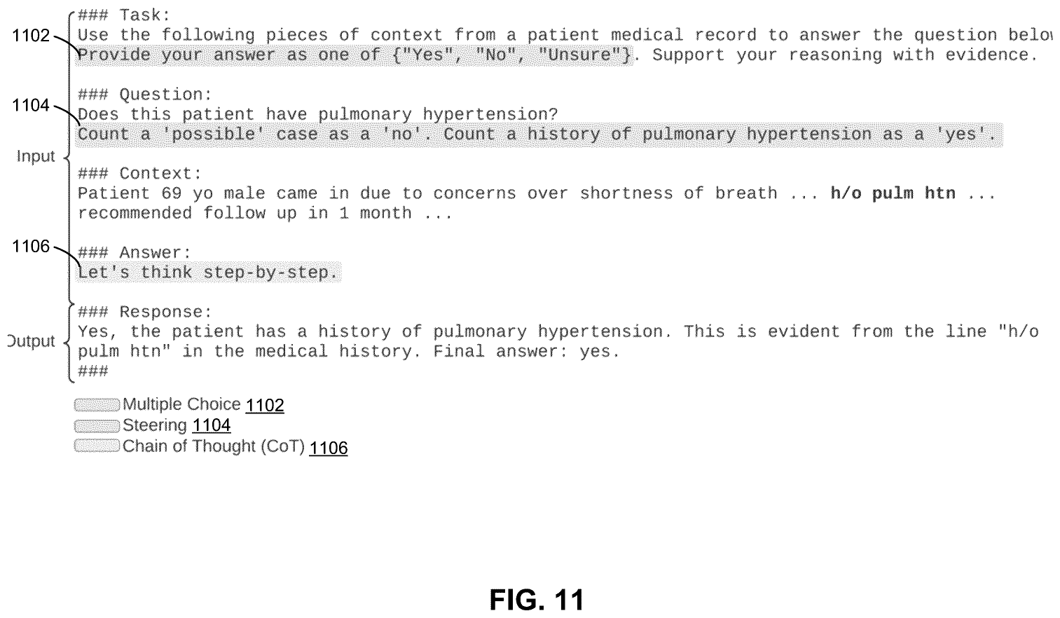

Accordingly, in some embodiments, each identified snippet (e.g., identified snippets 804, as illustrated in FIG. 8) is presented as context to the LLM, along with a set of instructions to facilitate decision-making. In some embodiments, the LLM is asked through a prompt to indicate whether the snippet indicates that the subject has PH. An example of a prompt asking an LLM to evaluate whether EHR snippets indicate pulmonary hypertension (PH) is shown in FIG. 11.

In some embodiments, the prompt instructs the LLM to answer in a yes or no form, or in a yes, no, or uncertain form. In some embodiments, the prompt further instructs the LLM to support its answer with evidence. In some embodiments, by prompting the LLM to support its answer with evidence, the LLM will essentially summarize the relevant portion of the snippet, reducing the context that will be fed into a second LLM (e.g., in a map-reduce LLM chain).

In some embodiments, the prompt includes a statement that steers the LLM. For example, referring again to the example of phenotyping for pulmonary hypertension, the prompt may instruct the LLM to count a ‘possible’ case of PH as ‘no’ answer. In some embodiments, the prompt instructs the LLM to count a clinical note of a history of PH as a ‘yes’ answer. In some embodiments, the LLM is further provided with examples of evidence that indicate the presence of the target medical condition. In some embodiments, the LLM is further provided with examples of evidence that do not indicate the presence of the target medical condition. In some embodiments, the LLM is further provided with evidence that indicate the absence of the target medical condition.

In some embodiments, the prompt includes a Chain-of-Thought (CoT) phrase. Use of CoT enhances reasoning by LLMs.

Outputs (e.g., outputs 806, as illustrated in FIG. 8) generated for individual snippets by the LLM are then aggregated to formulate the final decision (e.g., decision 808, as illustrated in FIG. 8). In some embodiments, the aggregation is performed using an LLM. In some embodiments, the LLM is provided the outputs from the snippet evaluation as context and is provided the same instructional prompts as for the evaluation of the individual snippets. In some embodiments, the LLM is provided the outputs from the snippet evaluation as context but is provided different instructional prompts as for the evaluation of the individual snippets. For example, the LLM may be asked whether any of the outputs from the snippet evaluation indicate a positive diagnosis for the target medical condition. In some embodiments, the aggregation is a max aggregation function, which checks if any of the individual snippet queries returned a positive diagnosis and, if so, assigns a positive label to the patient as whole.

In some embodiments, the snippet evaluation and aggregation step are performed using the same LLM. In some embodiments, the snippet evaluation and aggregation steps are performed by the LLM after a single prompt asking the LLM whether the subject has the medical condition based on evidence contained within the snippets. In some embodiments, the snippet evaluation and aggregation step are performed in series, such that the LLM is provided separate prompts for the two steps. In some embodiments, the snippet evaluation and aggregation step are performed using different LLMs.

In some embodiments, the methods described herein are processed through APIs that interface with an EHR database and/or AI component. In some embodiments, a user prompt is received at an API with instructions to retrieve snippets and then present them to an AI component responsive to a user prompt. In some embodiments, the API receives a prompt relating to a first subject or group of subjects. In some embodiments, medical records for the subject or group of subjects have already been parsed (snippetized) and snippets saved to a curated database. In some embodiments, the snippetized records have also been sorted to identify snippets related to a target medical condition, e.g., in the curated database. In some such cases, the API retrieves the presorted snippets from the database and presents them to an AI component. In other embodiments, where the medical records have not been snippetized, the API retrieves the medical record and directs a module (e.g., a natural language processing module) to parse the medical record into snippets and optionally sort the snippets to identify those snippets related to the target medical condition. Similarly, in some embodiments where the medical records have been snippetized but have not been sorted, the API retrieves the snippets and directs a module (e.g., a natural language processing module) to identify those snippets related to the target medical condition. The API then presents the identified snippets to the AI component (e.g., an LLM) in parallel (e.g., via separate instances of the AI component) or sequentially and asks the AI component whether each snippet indicates that the subject has the target medical condition, and optionally to provide reasoning for the answer. The AI component generates answers for each of the snippets and optionally the secondary logic (reasoning) for each answer. The API also includes instructions for aggregating the component answers into a final answer as to whether the subject has the target medical condition. In some embodiments, the API asks the LLM to aggregate the component answers, and optional secondary logic, such that the AI component may not provide component answers externally, but rather returns a single answer for the subject, which is returned as the response to the API prompt containing the query.

### Example Embodiments

Various example embodiments and aspects of the disclosure are described below for convenience. These are provided as examples, and do not limit the subject technology. Some of the examples described below are illustrated with respect to the figures disclosed herein simply for illustration purposes without limiting the scope of the subject technology.

(A1) In one aspect, some embodiments include a method of phenotyping (e.g., the method 600). In some embodiments, the method is performed at a computing system (e.g., the platform 100, the client device 102, or the server system 106). The method includes: (i) receiving a request to identify a target population (e.g., having one or more predefined characteristics); (ii) identifying a set of subjects as potential members of the target population; (iii) obtaining subject information (e.g., medical information) for the set of subjects (e.g., using a retriever component); (iv) providing the subject information to an artificial intelligence (AI) component (e.g., a generative AI component); (v) providing a set of natural language instructions to the A1 component, where the set of natural language instructions instruct the AI component how to determine if a subject belongs to (e.g., is a member of) the target population; and (vi) obtaining, from the AI component, identification of a subset of subjects from the set of subjects, the subset of subjects determined by the AI component to be members of the target population (e.g., determined to have the one or more predefined characteristics). In some embodiments, statistics about the subset of subjects are derived and provided to the user (e.g., instead of the identification of the subset of subjects). In some embodiments, the request is received from a client device. In some embodiments, the request is received via a user interface (e.g., the user interface 304). In some embodiments, the AI component is a component of the assistant module 226 and/or the assistant module 316. In some embodiments, the request includes inclusion and/or exclusion criteria for the target population.

In some embodiments, the request to identify the target population comprises a request to identify a target population having a phenotype. In some embodiments, the one or more predefined characteristics include subject characteristics (e.g., height, weight, gender, age, eye color, and/or blood type), subject condition and/or disease state, and/or treatment history. In some embodiments, the set of subjects (e.g., 1,000 or more, 10,000 or more, or 100,000 or more subjects) are identified from a pool of 1 million or more subjects (e.g., using regex search, BM25 search, and/or sparse vector search). In some embodiments, the subset of subjects includes 100 subjects or more, 1000 subjects or more, or 10,000 subjects or more. In some embodiments, the AI component is configured to exclude subjects from the subset of subjects based on the subject information. For example, the AI component may exclude subjects that (i) have a correct diagnosis and mutation for inclusion criteria, but did not receive expected therapy, (ii) have positive biomarker result, but have a medication planned rather than administered, (iii) have correct diagnosis, but not during inclusion criteria time period.

In some embodiments, the set of subjects are identified by searching a first set of databases (e.g., searching patient records in the database(s)). In some embodiments, the subject information is obtained by searching a second set of databases (e.g., using subject ids for the set of subjects). In some embodiments, the first set of databases includes a same database as the second set of databases. In some embodiments, the set of natural language instructions provide a context to the A1 component for natural language processing of the corresponding medical information to determine whether a respective subject in the set of subjects has at least one of the one or more predefined characteristics. In some embodiments, obtaining the identification of the subject of subjects comprises obtaining, from the AI component, identification of subjects from the set of subjects determined by the AI component to be, or to have a high likelihood of being, a member of the target population through a determination by the AI component that each subject in the subset of subjects has at least one of the one or more predefined characteristics, where, for each respective subject in the subset of subjects, the determination for at least one of the one or more predefined characteristics is made through natural language processing of corresponding medical information using the set of natural language instructions.

The phenotyping described herein allows for phenotyping difficult and/or rare diseases without subject matter expert (SME) created rules. As an example, an AI component (e.g., including an LLM) is prompted to identify a subject having a particular disease. The LLM-prompting approach can reduce/eliminate the SME knowledge translation problem, may not require training an ML model (e.g., zero-shot), and improves the phenotype development time (e.g., time-to-market). The LLM-prompting approach may be more robust than other phenotyping techniques, which often rely on a limited number of codes, modalities, or data elements (such as focusing only on diagnostic codes or procedures). For example, many studies, especially in early observational research or less methodologically rigorous studies, use only a single ICD code or a limited number of codes.

(A2) In some embodiments of A1, the target population comprises a target cohort (e.g., having a first medical condition) or having experienced an outcome of interest.

(A3) In some embodiments of A1 or A2, the AI component comprises a large language model (LLM). In some embodiments, the AI component comprises one or more agents (e.g., the agent(s) 318). In some embodiments, a prompt template is filled with the patient/medical information (obtained via a retriever model) and instructs the AI component in how to decide on the subject categorization. The instruction(s) may contain intermediate information, such as the LLM's reasoning and previous answers.

(A4) In some embodiments of A3, the LLM is not trained for phenotyping or identifying candidate subjects prior to obtaining the identification of the subset of subjects. Other approaches to encode SME-level decision making into an algorithm, to overcome the human labeling bottleneck problem, include training the LLM. However, training an LLM has certain drawbacks. Probabilistic, unsupervised, weak label phenotypes (e.g., LEVI/HOBBES) reduce cycle time compared to a manual method by offloading aggregation to an ML model, but at the cost of a lack of supervision. LLM-based supervised phenotypes (e.g., PALMER) provide supervision and highly flexible parameterization, but are resource intensive (e.g., computation, time, and labels).

In some embodiments, the LLM is subjected to instruction fine-tuning. For example, the LLM may be trained to follow a wide variety of instructions/prompts and can generalize this capacity across a wide number of tasks. In some embodiments, the LLM is configured as a reasoning agent. For example, by carefully crafting prompts, the LLM may be instructed to form a task, such as generating code, summarizing a document, or creating a form letter. Further, these tasks can be performed by the LLM without needing to re-train the LLM, e.g., the tasks can be performed zero-shot.

(A5) In some embodiments of any of A1-A4, the set of natural language instructions include one or more instructions to prevent hallucinations by the generative AI component.

(A6) In some embodiments of any of A1-A5, the set of subjects are identified from a patient database (e.g., the database(s) 400) using one or more filters. In some embodiments, the set of subjects are identified using a regex search, a BM25 search, and/or a sparse vector search.

(A7) In some embodiments of any of A1-A6, the set of subjects are identified based on patient data and patient file metadata. In some embodiments, the patient data includes an EHR.

(A8) In some embodiments of any of A1-A7, the retriever component is configured to identify candidate notes from patient files. For example, a retriever component may search a patient index to identify the data relevant to the task at hand. The retriever component may generate regular expression (regex) queries, sparse vector searches (such as term frequency-inverse document frequency (TF-IDF), bag-of-words retrieval (e.g., BM25), BM25+EC (elastic-search), and/or sparse neural search (e.g., SPLADE)), and/or dense vector searches (such as custom embedding models and/or sentence transformers). The top k results may be surfaced and potentially re-ranked to then be iteratively fed as context in a prompt template for the AI component.

(A9) In some embodiments of any of A1-A8, the method further includes indexing a database of patient files, where the medical information is obtained from the indexed database of patient files.

(A10) In some embodiments of A9, the method further includes generating a set of embeddings from the database of patient files, where the set of subjects is identified using the set of embeddings. In some embodiments, patient information (e.g., clinical notes, attachments, and/or EHR) is stored in a database that has regular expression (regex) search capability, and a retriever model (or other component) uses regex to obtain patient information (e.g., medical information) for each patient. In some embodiments, a vector index is generated from the patient information using an embedding model. In some embodiments, an AI component (e.g., an LLM) is provided with the (full) patient information (e.g., no retriever model is used).

(A11) In some embodiments of any of A1-A10, identifying the set of subjects includes obtaining respective identifiers for the set of subjects, and the medical information is obtained using the identifiers.

(A12) In some embodiments of any of A1-A11, the request to identify the target population is received from a user. In some embodiments, the method further includes providing the identification of the subset of subjects to the user. In some embodiments, the method further includes providing information about the subset of the subjects to the user (e.g., statistics or characterizations about the subset of the subjects).

(A13) In some embodiments of any of A1-A12, identifying the set of subjects includes: (i) generating a query embedding from the request to identify the target population; (ii) identifying one or more embeddings in a database of patient information that are similar to the query embedding; and (iii) determining that the one or more embeddings correspond to the set of subjects. For example, the one or more embeddings may be identified using a k-nearest neighbors (KNN) algorithm.

(A14) In some embodiments of any of A1-A13, the method further includes providing the identification of the subset of subjects to a user. In some embodiments, the method further includes providing information about the subset of the subjects to the user (e.g., statistics or characterizations about the subset of the subjects).

(A15) In some embodiments of any of A1-A14, the request provides a list of characteristics associated with the phenotype. In some embodiments, the list of characteristics includes the one or more predefined characteristics. In some embodiments, the list of characteristics includes only a subset of the one or more predefined characteristics.

(A16) In some embodiments of any of A1-A15, at least a subset of the one or more predefined characteristics are obtained via a look-up table or through a search of a medical reference. For example, at least a subset of the one or more predefined characteristics may be obtained from a knowledge database (e.g., the knowledge database 404).

(A17) In some embodiments of any of A1-A16, the medical information includes one or more of: an age, a gender, a cancer stage, a tumor size, an indication of lymph node involvement, a metastasis status, a hormone receptor status, a HER2 status, a cancer type, a cancer location, a therapy, a fatigue status, a vital status, and a laboratory result.

(A18) In some embodiments of any of A1-A17, the one or more predefined characteristics includes a first characteristic that is a predefined treatment regimen incurred and a second characteristic that is a biomarker status. In some embodiments, the one or more predefined characteristics are obtained using a knowledge translation model with the phenotype information inputted.

(A19) In some embodiments of any of A1-A18, the medical information for a subject in the set of subjects includes first data in a first format (e.g., natural language text) and second data in a second format (e.g., structured marker results).

(A20) In some embodiments of A19, the first format is an electronic health record format, and the second format is molecular data independent of the first format.

(A21) In some embodiments of any of A1-A20, the one or more predefined characteristics is a plurality of characteristics, and a first characteristic in the plurality of characteristics is treatment with a drug from the group consisting of sunitinib, lestaurtinib, midostaurin, crenolanib, gliteritinib, and sorafenib.

(A22) In some embodiments of any of A1-A21, the AI component includes a plurality of parameters, and obtaining the identification of the subset of subjects includes inputting into the AI component the medical information of a first subject in the set of subjects thereby obtaining, as output from the AI component, a determination as to whether the first subject includes the one or more predefined characteristics, by application of the medical information of the first subject to the plurality of parameters.

(A23) In some embodiments of any of A1-A21, the AI component includes a plurality of more parameters, and obtaining the identification of the subset of subjects includes inputting into the AI component the medical information of a first subject in the set of subjects thereby obtaining, as output form the AI component, a determination as to likelihood that the first subject includes the one or more predefined characteristics, by application of the medical information of the first subject to the plurality of parameters.

(A24) In some embodiments of A22 or A23, the plurality of parameters comprises 1000 or more parameters, 10,000 or more parameters, or 1×10⁶ or more parameters.

(A25) In some embodiments of any of A1-A24, at least a portion of the medical information for a subject in the set of subjects is treated as unstructured by the AI component.

(A26) In some embodiments of A25, the set of natural language instructions provides a first context for interpreting a first portion of medical information for a subject in unstructured form and a different second context for interpreting a second portion of the medical information in unstructured form. For example, the set of natural language instructions may provide certain context for diagnosis and provide different certain context for marker results in molecular results.

(A27) In some embodiments of any of A1-A26, less than forty percent, less than thirty percent, or less than twenty percent of the set of subjects are determined by the AI component to have the one or more predefined characteristics.

(A28) In some embodiments of any of A1-A27, the set of subjects includes 100 or more, 1000 or more, or 10,000 or more subjects.

(B1) In another aspect, some embodiments include a method of phenotyping. In some embodiments, the method is performed at a computing system (e.g., the platform 100, the client device 102, or the server system 106). The method includes: (i) receiving a request to phenotype a patient with respect to a medical condition; (ii) retrieving a plurality of snippets corresponding to text in a medical record for the subject; (iii) providing to an artificial intelligence (AI) component (i) each respective snippet in the plurality of snippets, and (ii) a set of natural language instructions, wherein the set of natural language instructions provide a context to the AI component for natural language processing of each respective snippet in the plurality of snippets to obtain, as output from the AI component, for each respective snippet, a corresponding answer as to whether the respective snippet indicates the subject has the medical condition, thereby generating a plurality of answers; and (iv) aggregating the plurality of answers to determine the phenotype of the subject.

(B2) In some embodiments of B1, the retrieving the plurality of snippets comprises inputting each respective snippet in a set of precursor snippets corresponding to text in the medical record for the subject into a retriever model to determine whether the respective snippet contains information related to the medical condition and retrieving those respective snippets in the set of precursor snippets determined to contain information related to the medical condition.

(B3) In some embodiments of B2, the retriever model comprises pattern matching of one or more regular expressions related to the medical condition.

(B4) In some embodiments of any of B1-B3, the AI component comprises a large language model (LLM).

(B5) In some embodiments of any of B1-B4, the AI component is not trained for phenotyping with respect to the medical condition.

(B6) In some embodiments of any of B1-B5, each respective snippet in the plurality of snippets is provided to a corresponding instance of the AI component in parallel.

(B7) In some embodiments of any of B1-B6, the set of natural language instructions comprises an instruction to provide a discrete answer as to whether the respective snippet indicates the subject has the medical condition.

(B8) In some embodiments of B7, the discrete answer is selected from yes, no, and unsure.

(B9) In some embodiments of any of B1-B8, the set of natural language instructions comprises an instruction to provide a reasoning for the corresponding answer.

(B10) In some embodiments of any of B1-B9, the set of natural language instructions comprises prompt that steers the AI component to provide a first answer when a first condition is met.

(B11) In some embodiments of any of B1-B10, the set of natural language instructions comprises a chain-of-thought (CoT) prompt.

(B12) In some embodiments of any of B1-B11, each respective snippet in the plurality of snippets is provided to a corresponding instance of the AI component in parallel.

(B13) In some embodiments of any of B1-B12, the aggregating comprises evaluating a max aggregation function that returns a positive phenotype for the medical condition when at least one corresponding answer indicates the subject has the medical condition.

(B14) In some embodiments of any of B1-B13, the aggregating comprises providing to a second artificial intelligence (AI) component (i) each corresponding answer, and (ii) a set of natural language instructions, wherein the set of natural language instructions provide a context to the AI component for determining, based on each corresponding answer, as final answer as to whether the subject has the medical condition.

(B15) In some embodiments of any of B1-B14, retrieving the plurality of snippets comprises identifying respective snippets in a set of precursor snippets determined to contain information related to the medical condition, ranking the identified snippets, and retrieving a subset of identified snippets satisfying a ranking threshold.

In another aspect, some embodiments include a computing system (e.g., the platform 100, the client device 102, or the server system 106) including control circuitry (e.g., the CPUs 302) and memory (e.g., the memory 310) coupled to the control circuitry, the memory storing one or more sets of instructions configured to be executed by the control circuitry, the one or more sets of instructions including instructions for performing any of the methods described herein (e.g., A1-A28 and B1-B15 above).

In yet another aspect, some embodiments include a non-transitory computer-readable storage medium storing one or more sets of instructions for execution by control circuitry of a computing system, the one or more sets of instructions including instructions for performing any of the methods described herein (e.g., A1-A28 and B1-B15 above).

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. In some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥ 4,000; n≥5,000; n≥7,500; n≥10,000; n≥20,000; n≥40,000; n≥75,000; n≥100,000; n≥ 200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “instruction” refers to an order given to a computer processor by a computer program. On a digital computer, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

In some embodiments, the methods described herein include inputting information into a model comprising a plurality of parameters, wherein the model applies the plurality parameters to the information through a plurality of instructions to generate an output from the model.

In some embodiments, the plurality of parameters is at least 1000 parameters, at least 5000 parameters, at least 10,000 parameters is at least 50,000 parameters, at least 100,000 parameters, at least 250,000 parameters, at least 500,000 parameters, at least 1 million parameters, at least 5 million parameters, at least 10 million parameters, at least 25 million parameters, at least 50 million parameters, at least 100 million parameters, at least 250 million parameters, at least 500 million parameters, at least 1 billion parameters, or more parameters.

In some embodiments, the plurality of instructions is at least 1000 instructions, at least 5000 instructions, at least 10,000 instructions is at least 50,000 instructions, at least 100,000 instructions, at least 250,000 instructions, at least 500,000 instructions, at least 1 million instructions, at least 5 million instructions, at least 10 million instructions, at least 25 million instructions, at least 50 million instructions, at least 100 million instructions, at least 250 million instructions, at least 500 million instructions, at least 1 billion instructions, or more instructions.

### Examples

To evaluate a retrieval-augmented generative (RAG) approach to zero-shot phenotyping using LLMs, in accordance with some implementations described herein, an unseen dataset of EHRs was processed to identify instances of pulmonary hypertension (PH). PH is a cardiopulmonary condition characterized by abnormally elevated pressure in the arteries of the lung and right side of the heart. It is driven by multiple underlying etiologies and is typically characterized by 5 subgroups. While the prevalence of specific PH etiologies may vary across subgroups, it is broadly categorized as a rare disease with an estimated global prevalence rate of 1-3%. It is often underdiagnosed or diagnosed too late, leading to limited treatment options and poor prognosis. Building a PH phenotype is complicated by the fact that the hemodynamic definition of PH has changed over time. PH was formerly characterized by a mean pulmonary artery pressure (mPAP)≥25 mm Hg measured by right heart catheterization (RHC); however, in 2018 the definition adopted a new threshold of mPAP>20 mm Hg. This means that there are some patients who would be currently identified as having PH under the new definition whereas during the time of their original treatment or workup they would not have been diagnosed with PH. The ability to systematically identify PH patients who would otherwise not be identified could significantly impact patient outcomes.

The dataset was made-up of de-identified clinical notes from a large hospital serving a population of several million patients. Given the expected low prevalence rate of PH within this population, an enriched cohort of patients displaying any clinical evidence of PH in either the structured data or clinical notes was identified. From this cohort, several hundred patients were randomly selected for a comprehensive chart review. Each patient underwent independent evaluation by two physicians, with any discrepancies resolved through joint discussion to reach a consensus. The physicians regarded a diagnosis based on RHC findings as the gold standard for diagnosing PH. Subsequently, these labeled patients were divided into three groups: a training set, a validation set, and a test set, each of which had approximately the same distribution of positive PH cases and negative controls.

### RAG Phenotyping

Briefly, the unstructured clinical notes from each EHR in the dataset were tokenized and then divided into snippets of 2,048 “tokens” in size. Regex was then used to identify relevant snippets. The Regex rules encompassed a broad spectrum of patterns that could potentially be associated with PH.

All retrieved patient snippets were then fed into the LLM bison@001, which is a version of PaLM-2 available through Google Cloud's Vertex-AI offering. PaLM-2 builds upon the foundation of PaLM-1, incorporating a combination of various pre-training objectives to achieve state-of-the-art results on several benchmarks while maintaining smaller model sizes. Each snippet was concurrently presented as context to the LLM, along with a set of instructions to facilitate decision-making and provide reasoning for the decision.

The outputs generated from the LLM evaluation of the snippets (one per snippet) were then aggregated to formulate a final decision as to whether the patient has ever had pulmonary hypertension. Two different aggregation approaches were evaluated for aggregating: (1) an LLM-based approach, which aggregates the outputs and reasoning from each individual snippet query into a larger prompt for a final decision by an LLM through prompting (Max Aggregation); and (2) a Max aggregation function, which checks if any of the individual snippet queries returned a positive diagnosis and, if so, assigns a positive label to the patient as whole. In the LLM aggregation approach, two different variations were evaluated: (1) applying the same prompt that was provided at the snippet level to aggregate responses (LLM—Same Prompt); and (2) applying a different prompt that asks the LLM if any of the responses indicated a positive diagnosis (LLM—Different Prompt). An example aggregation input prompt and LLM output is illustrated in FIG. 11.

Echocardiogram (ECHO) and computerized tomography (CT) studies are commonly performed on patients suspected of having PH, with reports typically generated by technicians or clinicians that may mention the presence of PH. However, these reports alone are not sufficient for a clinical diagnosis because they may not be confirmed by a physician. Moreover, it is known that ECHO and CT have higher error rates when compared to the gold standard of RHC.

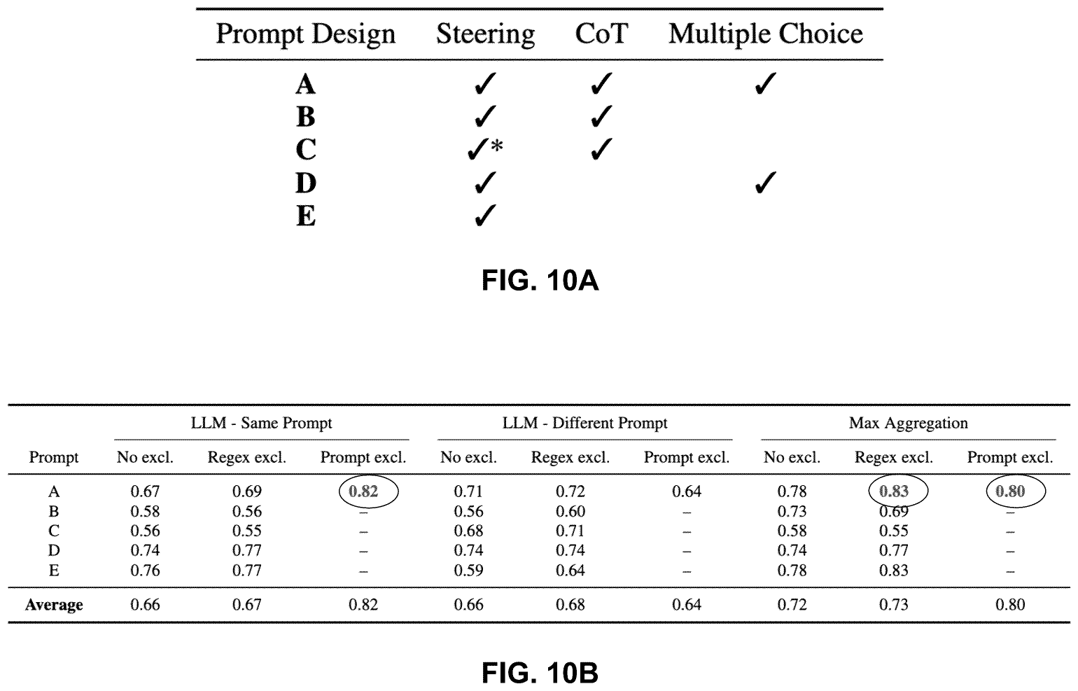

While reviewing model errors on the validation set, a significant number of false positives were identified as originating from echocardiography reports noting suspicion of PH without any confirmatory diagnostic testing nor a clinical diagnosis by a provider. As these reports typically exhibit a consistent structure, it was explored whether these snippets could be extracted from these technician reports in two ways: (1) employing regular expressions to filter out snippets containing headers and common language found in these reports and (2) updating LLM prompts to instruct the LLM to disregard ECHO and CT reports. As reported in FIG. 10B, excluding these snippets improved performance of the model when evaluated against the validation set.

Several iterations of prompt design, snippet exclusions, and aggregation methods were evaluated. Briefly, various zero-shot prompt designs to query the LLM for the diagnosis of PH were explored. Additionally, the value of Chain-of-Thought (CoT) reasoning by enhancing the prompt with the phrase “let's think step-by-step” was evaluated. Finally, prompts were used to guide the model to consider possible cases of PH as negative diagnosis and history of PH as positive diagnosis. In total, 5 different prompt designs were tested, as outlined in FIG. 10A. These prompt designs were further used in combination with the three aggregation methodologies described above, with either no exclusion of Echocardiogram (ECHO) and computerized tomography (CT) reports, exclusion of ECHO and CT reports at the Regex step, or exclusion of ECHO and CT reports through LLM prompts. FIG. 10B shows the performance of these different permutations of the LLM-based phenotype pipeline over the validation set, measured in terms of F1 score. LLM-based aggregation methods, whether using the same or different prompts at the snippet level, resulted in average F1 scores of 0.67 and 0.68, respectively, with significant variability in performance across choice of prompting design. In contrast, Max aggregation achieved an F1 score of 0.73, also providing increased stability across different prompts. The steering column refers to steering the LLM to count history of PH as a ‘no’ and possibly having PH as a ‘yes’. In template C, the prompt was modified to remove the phrase “explain your reasoning,” which is included in all other prompt templates.

A fair amount of variability in performance was across these different prompt designs without any prominent features defining those prompts that appeared to perform best. Therefore, the three highest performing designs, indicated by circled results in FIG. 10B, were selected for comparison with a structured phenotype, rules-based algorithm.

### Rules-Based Phenotyping Algorithm

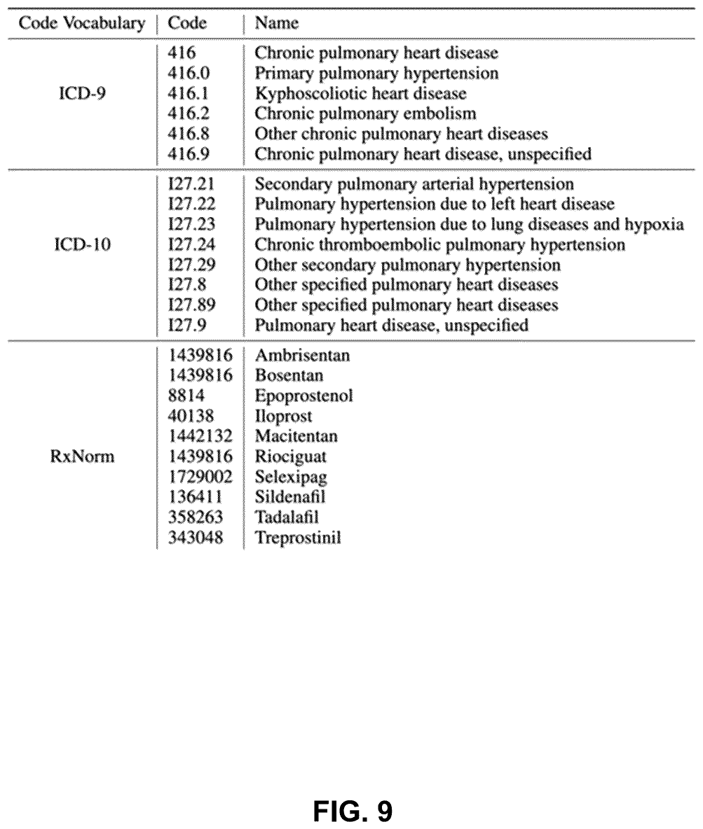

To compare the RAG-based LLM phenotyping described above to conventional rules-based phenotyping, the same dataset enriched for instances of PH was phenotyped using a rules-based structured phenotype. Briefly, a physician conducted a review of patients within the training dataset to establish a rules-based algorithm for diagnosing PH using EHRs. Following a thorough examination of the literature on PH phenotypes, the rules encompassed a blend of ICD-9/10 code frequencies, medication records, laboratory data, and other clinical features available in the patients' records. After a series of iterative reviews and adjustments to the model output, the physician ceased further model development when the incremental improvements began to diminish. The diagnostic and medication codes that make up the structured phenotype for PH are shown in FIG. 9.

### Comparison of RAG-Based LLM Phenotyping to Rules-Based Structured Phenotyping

Table 1 compares the performance of the three variations of the LLM-based phenotyping architecture with the structured phenotype baseline developed by a physician on the test set. As demonstrated, LLM-based phenotypes generally show improvements between 18% and 21% over the structured phenotype. There was no notable drop in F1 scores, ranging from 0.05 to 0.1, in the performance of LLM-based methods compared to the results obtained on the validation set, which might be attributed to the larger evaluation cohort and potentially to some overfitting on the training set. Nevertheless, the LLM-based methods significantly outperformed the structured phenotype method, resulting in the identification of approximately twice as many patients with a confirmed diagnosis of PH. In a real-world application, these patients might otherwise remain undiagnosed.

TABLE 1
Performance of three different variants of the LLM-based phenotype
architecture compared with that of the structured
phenotype developed by a physician on the test set.

| Model | Aggregation Technique | ECHO Exclusion | F1 Score |
|---|---|---|---|
| Structured | N/A | N/A | 0.62 |
| LLM | Max | Regex | 0.73 |
| LLM | Max | Amended Prompt | 0.75 |
| LLM | LLM | Amended Prompt | 0.72 |

Furthermore, as reported in Table 2, it was observed that the retrieved documents spanned 29 distinct note types, highlighting the importance of retrieving across note types to accurately identify disease diagnoses.

TABLE 2
Distribution of note types retrieved by the LLM phenotyping pipeline
within the test set. “Other” consists of 23 different note types.

| Note Type | Frequency |
|---|---|
| Progress Note | 54% |
| Consult | 8% |
| Discharge Summary | 6% |
| History & Physical Exam | 6% |
| Procedure | 5.5% |
| Telephone Encounter | 4% |
| Other | 16.5% |

All references cited herein are incorporated herein by reference in their entirety and for all purposes to the same extent as if each individual publication or patent or patent application was specifically and individually indicated to be incorporated by reference in its entirety for all purposes.

Another aspect of the present disclosure provides a computer system comprising one or more processors, and a non-transitory computer-readable medium including computer-executable instructions that, when executed by the one or more processors, cause the processors to perform a method according to any one of the embodiments disclosed herein, and/or any combinations, modifications, substitutions, additions, or deletions thereof as will be apparent to one skilled in the art.

Another aspect of the present disclosure provides a non-transitory computer-readable storage medium having stored thereon program code instructions that, when executed by a processor, cause the processor to perform the method according to any one of the embodiments disclosed herein, and/or any combinations, modifications, substitutions, additions, or deletions thereof as will be apparent to one skilled in the art.

The present invention can be implemented as a computer program product that comprises a computer program mechanism embedded in a non-transitory computer readable storage medium. For instance, the computer program product could contain the program modules shown in any combination in FIG. 1 and/or as described elsewhere within the application. These program modules can be stored on a CD-ROM, DVD, magnetic disk storage product, USB key, or any other non-transitory computer readable data or program storage product.

Many modifications and variations of this disclosure can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The disclosure is to be limited only by the terms of the appended claims, along with the full scope of equivalents to which such claims are entitled.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purposes of explanation, has been described with reference to specific embodiments. However, the illustrative discussions above are not intended to be exhaustive or limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A method of phenotyping performed at a computing system, comprising:
receiving a request to identify a target population having a phenotype;
obtaining one or more predefined characteristics associated with the phenotype;
identifying, using a retriever model, a set of subjects as potential members of the target population by searching a first set of one or more databases for patient records;
obtaining, for each respective subject in the set of subjects, medical information by searching a second set of one or more databases;
providing, for each respective subject in the set of subjects, corresponding medical information of the medical information to an artificial intelligence (AI) component;
providing a set of natural language instructions to the AI component, wherein the set of natural language instructions provide a context to the AI component for natural language processing of the corresponding medical information to determine whether a respective subject in the set of subjects has at least one of the one or more predefined characteristics; and
obtaining, from the AI component, identification of a subset of subjects from the set of subjects determined by the AI component to be, or to have a high likelihood of being, a member of the target population through a determination by the AI component that each subject in the subset of subjects has at least one of the one or more predefined characteristics, wherein, for each respective subject in the subset of subjects, the determination for at least one of the one or more predefined characteristics is made through natural language processing of corresponding medical information using the set of natural language instructions.
